(19) United States
(12) Patent Application Publication
(10) Pub. No.: US 2009/0158465 A1
(43) Pub. Date: Jun. 18, 2009

(54) TRANSGENIC PLANTS WITH ENHANCED DROUGHT-RESISTANCE AND METHOD FOR PRODUCING THE PLANTS

(75) Inventors: Zhizhong Gong, Beijing (CN); XuHui Hong, Beijing (CN)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: D-Helix, San Francisco, CA (US)

(21) Appl. No.: 11/846,425

(22) Filed: Aug. 28, 2007

Related U.S. Application Data
(60) Provisional application No. 60/841,504, filed on Aug. 31, 2006.

(51) Int. Cl. C12N 15/29; A01H 5/00; C12N 15/00

(52) U.S. Cl. 800/287; 800/278; 800/298; 435/320.1

(57) ABSTRACT

Mutation of ELO2 is newly demonstrated to result in plants with increased sensitivity to abscisic acid (ABA).

### CROSS-REFERENCES TO RELATED APPLICATIONS

The present patent application claims benefit of priority to U.S. Provisional Patent Application No. 60/841,504, filed Aug. 31, 2006, which is incorporated by reference in its entirely for all purposes.

### FIELD OF THE INVENTION

This invention relates to methods and compositions for generating plants with altered abscisic acid sensitivity.

### BACKGROUND OF THE INVENTION

The phytohormone abscisic acid (ABA) regulates many agriculturally important stress and developmental responses throughout the life cycle of plants. In seeds, ABA is responsible for the acquisition of nutritive reserves, desiccation tolerance, maturation and dormancy (M. Koornneef et al., Plant Physiol. Biochem., 36:83 (1998); J. Leung & J. Giraudat, Annu. Rev. Plant. Physiol. Plant. Mol. Biol., 49:199 (1998)). During vegetative growth, ABA is a central internal signal that triggers plant responses to various adverse environmental conditions including drought, salt stress and cold (M. Koornneef et al., Plant Physiol. Biochem., 36:83 (1998); J. Leung & J. Giraudat, Annu. Rev. Plant. Physiol. Plant. Mol. Biol., 49:199 (1998)). A rapid response mediated by ABA is stomatal closure in response to drought (J. Leung & J. Giraudat, Annu. Rev. Plant. Physiol. Plant. Mol. Biol., 49:199 (1998); E. A. C. MacRobbie, Philos. Trans. R Soc. Lond. B Biol. Sci., 353:1475 (1998); J. M. Ward et al., Plant Cell, 7:833 (1995)). Stomata on the leaf surface are formed by pairs of guard cells whose turgor regulates stomatal pore apertures (E. A. C. MacRobbie, Philos. Trans. R Soc. Lond. B Biol. Sci., 353:1475 (1998); J. M. Ward et al., Plant Cell, 7:833 (1995)). ABA induces stomatal closure by triggering cytosolic calcium ([Ca²⁺_{cyt}) increases which regulate ion channels in guard cells (E. A. C. MacRobbie, Philos. Trans. R Soc. Lond. B Biol. Sci., 353:1475 (1998); J. M. Ward et al., Plant Cell, 7:833 (1995)). This response is vital for plants to limit transpirational water loss during periods of drought.

### BRIEF SUMMARY OF THE INVENTION

The present invention provides for methods of enhancing abscisic acid sensitivity in a plant. In some embodiments, the methods comprise introducing an recombinant expression cassette into a plant, wherein the expression cassette comprises a promoter operably linked to a polynucleotide comprising at least 20 contiguous nucleotides complementary or identical to a contiguous sequence in a cDNA encoding an endogenous polypeptide substantially (e.g., at least 50%) identical to SEQ ID NO:2, SEQ ID NO:4 or SEQ ID NO:6 in the plant, wherein the promoter is heterologous to the polynucleotide, thereby reducing expression of the polypeptide in the plant, wherein the plant has increased abscisic acid sensitivity compared to an otherwise identical plant lacking the expression cassette.

In some embodiments, the polynucleotide comprises at least 50 contiguous nucleotides complementary or substantially identical to a contiguous sequence in the cDNA. In some embodiments, the polynucleotide comprises at least 200 contiguous nucleotides complementary or substantially identical to a contiguous sequence in the cDNA. In some embodiments, the polypeptide is substantially identical (e.g., at least 95% identical) to a protein selected from the group consisting of SEQ ID NO:2, SEQ ID NO:4 and SEQ ID NO:6.

In some embodiments, the plant has improved drought tolerance compared to an otherwise identical plant lacking the expression cassette. In some embodiments, the polynucleotide comprises at least 20 contiguous nucleotides complementary or substantially identical to a contiguous sequence in SEQ ID NO: 1, SEQ ID NO:3, or SEQ ID NO:5.

In some embodiments, the promoter directs expression to guard cells of the plant. In some embodiments, the promoter is constitutive. In some embodiments, the promoter is inducible. In some embodiments, the promoter is tissue-specific.

The present invention also provides for recombinant expression cassettes comprising a promoter operably linked to a polynucleotide comprising at least contiguous 20 nucleotides complementary or identical to a contiguous sequence in a cDNA encoding an endogenous polypeptide substantially (e.g., at least 50% identical) to SEQ ID NO:2, SEQ ID NO:4 or SEQ ID NO:6 in the plant, wherein the promoter is heterologous to the polynucleotide, and wherein introduction of the expression cassette into a plant results in enhanced abscisic acid sensitivity in the plant compared to an otherwise identical plant lacking the expression cassette.

In some embodiments, introduction of the expression cassette into a plant results in improved drought tolerance in the plant compared to an otherwise identical plant lacking the expression cassette

In some embodiments, the polynucleotide comprises at least 50 contiguous nucleotides complementary or substantially identical to a contiguous sequence in the cDNA. In some embodiments, the polynucleotide comprises at least 200 contiguous nucleotides complementary or substantially identical to a contiguous sequence in the cDNA.

In some embodiments, the polypeptide is at least 95% identical to a protein selected from the group consisting of SEQ ID NO:2, SEQ ID NO:4 and SEQ ID NO:6.

In some embodiments, the polynucleotide comprise at least 20 contiguous nucleotides complementary or substantially identical to a contiguous sequence in SEQ ID NO:1, SEQ ID NO:3, or SEQ ID NO:5.

In some embodiments, the promoter directs expression to guard cells of the plant. In some embodiments, the promoter is constitutive. In some embodiments, the promoter is inducible. In some embodiments, the promoter is tissue-specific.

The present invention also provides transgenic plants comprising a recombinant expression cassette. In some embodiments, the recombinant expression cassette comprises a promoter operably linked to a polynucleotide comprising at least 20 contiguous nucleotides complementary or identical to a contiguous sequence in a cDNA encoding an endogenous polypeptide substantially identical (e.g., at least 50% identical) to SEQ ID NO:2, SEQ ID NO:4 or SEQ ID NO:6 in the plant, wherein the promoter is heterologous to the polynucleotide, and wherein the plant has enhanced abscisic acid sensitivity compared to an otherwise identical plant lacking the expression cassette.

In some embodiments, the plant has improved drought tolerance compared to an otherwise identical plant lacking the expression cassette.

In some embodiments, the polynucleotide comprises at least 50 contiguous nucleotides complementary or substantially identical to a contiguous sequence in the cDNA. In some embodiments, the polynucleotide comprises at least 200 contiguous nucleotides complementary or substantially identical to a contiguous sequence in the cDNA.

In some embodiments, the polypeptide is at least 95% identical to a protein selected from the group consisting of SEQ ID NO:2, SEQ ID NO:4 and SEQ ID NO:6.

In some embodiments, the plant has improved drought tolerance compared to an otherwise identical plant lacking the expression cassette.

In some embodiments, the polynucleotide comprise at least 20 contiguous nucleotides complementary or substantially identical to a contiguous sequence in SEQ ID NO:1, SEQ ID NO:3, or SEQ ID NO:5.

In some embodiments, the promoter directs expression to guard cells of the plant. In some embodiments, the promoter is constitutive. In some embodiments, the promoter is inducible. In some embodiments, the promoter is tissue-specific.

The invention also provides for any plant part from the transgenic plants of the invention. Examples of such plant parts include, but are not limited to: seeds, flowers, leafs and fruits.

### DEFINITIONS

The term “nucleic acid” or “polynucleotide” refers to a single or double-stranded polymer of deoxyribonucleotide or ribonucleotide bases read from the 5′ to the 3′ end, or an analog thereof.

The term “promoter,” as used herein, refers to a polynucleotide sequence capable of driving transcription of a coding sequence in a cell. Thus, promoters used in the polynucleotide constructs of the invention include cis-acting transcriptional control elements and regulatory sequences that are involved in regulating or modulating the timing and/or rate of transcription of a gene. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) gene transcription. A “plant promoter” is a promoter capable of initiating transcription in plant cells. A “constitutive promoter” is one that is capable of initiating transcription in nearly all tissue types, whereas a “tissue-specific promoter” initiates transcription only in one or a few particular tissue types.

The term “plant” includes whole plants, shoot vegetative organs and/or structures (e.g., leaves, stems and tubers), roots, flowers and floral organs (e.g., bracts, sepals, petals, stamens, carpels, anthers), ovules (including egg and central cells), seed (including zygote, embryo, endosperm, and seed coat), fruit (e.g., the mature ovary), seedlings, plant tissue (e.g., vascular tissue, ground tissue, and the like), cells (e.g., guard cells, egg cells, trichomes and the like), and progeny of same. The class of plants that can be used in the method of the invention is generally as broad as the class of higher and lower plants amenable to transformation techniques, including angiosperms (monocotyledonous and dicotyledonous plants), gymnosperms, ferns, and multicellular algae. It includes plants of a variety of ploidy levels, including aneuploid, polyploid, diploid, haploid, and hemizygous.

A polynucleotide sequence is “heterologous” to an organism or a second polynucleotide sequence if it originates from a foreign species, or, if from the same species, is modified from its original form. For example, when a promoter is said to be operably linked to a heterologous coding sequence, it means that the coding sequence is derived from one species whereas the promoter sequence is derived another, different species; or, if both are derived from the same species, the coding sequence is not naturally associated with the promoter (e.g., is a genetically engineered coding sequence, e.g., from a different gene in the same species, or an allele from a different ecotype or variety).

A polynucleotide “exogenous” to an individual plant is a polynucleotide which is introduced into the plant by any means other than by a sexual cross. Examples of means by which this can be accomplished are described below, and include Agrobacterium-mediated transformation, biolistic methods, electroporation, and the like. Such a plant containing the exogenous nucleic acid is referred to here as a T₁ (e.g., in Arabidopsis by vacuum infiltration) or R₀ (for plants regenerated from transformed cells in vitro) generation transgenic plant.

As used herein, the term “transgenic” describes a non-naturally occurring plant that contains a genome modified by man, wherein the plant includes in its genome an exogenous nucleic acid molecule, which can be derived from the same or a different plant species. The exogenous nucleic acid molecule can be a gene regulatory element such as a promoter, enhancer, or other regulatory element, or can contain a coding sequence, which can be linked to a heterologous gene regulatory element. Transgenic plants that arise from sexual cross or by selfing are descendants of such a plant.

An “expression cassette” refers to a nucleic acid construct that, when introduced into a host cell, results in transcription and/or translation of an RNA or polypeptide, respectively. Antisense or sense constructs that are not or cannot be translated are expressly included by this definition. In the case of both expression of transgenes and suppression of endogenous genes (e.g., by antisense, or sense suppression) one of skill will recognize that the inserted polynucleotide sequence need not be identical, but may be only “substantially identical” to a sequence of the gene from which it was derived. As explained below, these substantially identical variants are specifically covered by reference to a specific nucleic acid sequence.

“Decreased” ELO2 expression or activity refers to a reduction in the protein's expression or activity. An example of such reduced activity or expression includes the following: ELO2 expression or activity is decreased compared to the level in wild-type, non-transgenic control plants (i.e., the quantity of ELO2 activity or expression of the ELO2 gene is reduced) in at least one tissue (in other tissues, the expression could be the same, or also reduced). Reduction of expression can be, for example, at least 50%, 25%, 10%, 5% or less than in the control plant.

Two nucleic acid sequences or polypeptides are said to be “identical” if the sequence of nucleotides or amino acid residues, respectively, in the two sequences is the same when aligned for maximum correspondence as described below. The terms “identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence over a comparison window, as measured using one of the following sequence comparison algorithms or by manual alignment and visual inspection. When percentage of sequence identity is used in reference to proteins or peptides, it is recognized that residue positions that are not identical often differ by conservative amino acid substitutions, where amino acids residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated according to, e.g., the algorithm of Meyers & Miller, Computer Applic. Biol. Sci. 4:11-17 (1988) e.g., as implemented in the program PC/GENE (Intelligenetics, Mountain View, Calif., USA).

The phrase “substantially identical,” used in the context of two nucleic acids or polypeptides, refers to a sequence that has at least 25% sequence identity with a reference sequence. Alternatively, percent identity can be any integer from 25% to 100%. More preferred embodiments include at least: 25%, 30%, 35%, 40%, 45%, 50%, 55%, 60%, 65%, 70%, 75%, 80%, 85%, 90%, 95%, or 99%, compared to a reference sequence using the programs described herein; preferably BLAST using standard parameters, as described below. This definition also refers to the complement of a test sequence, when the test sequence has substantial identity to a reference sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection.

Examples of algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1977) Nucleic Acids Res. 25: 3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10⁻⁵, and most preferably less than about 10⁻²⁰.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine) can be modified to yield a functionally identical molecule. Accordingly, each silent variation of a nucleic acid which encodes a polypeptide is implicit in each described sequence.

As to amino acid sequences, one of skill will recognize that individual substitutions, in a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art.

The following six groups each contain amino acids that are conservative substitutions for one another:

### 1) Alanine (A), Serine (S), Threonine (T);

2) Aspartic acid (D), Glutamic acid (E);

### 3) Asparagine (N), Glutamine (Q);

### 4) Arginine (R), Lysine (K);

### 5) Isoleucine (I), Leucine (L), Methionine (M), Valine (V); and

### 6) Phenylalanine (F), Tyrosine (Y), Tryptophan (W).

(see, e.g., Creighton, Proteins (1984)).

As used herein, the term “drought-resistance” or “drought-tolerance,” including any of their variations, refers to the ability of a plant to recover from periods of drought stress (i.e., little or no water for a period of days). Typically, the drought stress will be at least 5 days and can be as long as 18 to 20 days or more, depending on, for example, the plant species.

### BRIEF DESCRIPTION OF THE DRAWINGS

FIG. 1 illustrates abo1-1 mutant plants are resistant to drought stress. Reduced wilting was observed for abo1 mutant plants during water stress treatment. Wild-type (WT) and abo1-1 plants were grown with sufficient water for (A) 3 or (B) 2 weeks, and then water was withheld for 11 days. (C) Water loss in detached leaves from 3-week-old plants placed in a room (40% rH) for 14 h. (D) Comparison of rates of water loss from detached leaves of the wild-type and abo1-1 plants. Water loss is expressed as the proportion of initial fresh weight. Values are means ±SE from 15 leaves for each of three independent experiments.

FIG. 2 illustrates stomatal closure in abo1-1 plants is hypersensitive to ABA (A and B) but not to darkness (C and D). Data represent the means ±SE from 30 stomata measured for each data point, from three independent experiments. WT, wild type. Bar, 10 μm.

FIG. 3 illustrates seedling growth of abo1-1 plants is hypersensitive to ABA. (A) Comparison of abo1-1 and wild-type (WT) seedlings grown on MS medium for 5 days and then transferred to MS medium containing different concentrations of ABA. The pictures were taken 7 days after transfer. (B) Comparison of relative root lengths between abo1-1 and wild-type plants. Values are means ±SE (n=30). (C) Seed germination of abo1-1 and wild-type plants. Wild-type and abo1-1 seeds were planted on MS agar medium (upper rows) or MS medium containing 0.1 μM ABA (lower rows). Plates were transferred to a growth chamber after 3-day stratification. The pictures were taken 7 days after transfer. (D) Seedling greening rates of abo1-1 and wild-type plants. Ratio of green seedlings to total seedlings was determined after plates for seed germination were cultured for 7 days. Values are means ±SE from three independent experiments, with 100 seeds used per genotype per data point for each experiment.

FIG. 4 illustrates seed germination and seedling growth of abo1 mutant plants are resistant to oxidative stress. (A) Five-day-old seedlings of abo1-1 and wild-type (WT) plants were transferred to MS medium or MS medium containing different concentrations of MV. The pictures were taken after 5 days. (B) Germination comparison of wild-type and abo1-1 plants in the presence of 1.2 μM MV. The pictures were taken after 10 days. (C) Effects of different concentrations of MV on seedling greening rates of abo1-1 and wild-type plants grown on MS medium containing different concentrations of MV after 10 days. (D) Comparison of MV effects on abo1 mutant and wild-type leaves. (G) Mature leaves were floated on 1 mM MV for 5 days. (E) Ion leakage analysis of leaves treated with 1 mM MV at different times. (F) Five-day-old seedlings of abo1-1 and wild-type plants were transferred to MS medium containing 10 μM Rose Bengal. Mature leaves were treated with 10 mM H2O2 for 5 days. The pictures were taken after 3 days. (H) Ion leakage analysis of H2O2 effects on abo1 mutant and wild-type leaves.

FIG. 5 illustrates expression of ABA- and stress-responsive genes in abo1-2 (M) and wild-type (W) seedlings. Three-week-old seedlings growing on agar plates were treated with 20 μM ABA for 3 h (ABA-3) or 5 h (ABA-5). A tubulin gene was used as a loading control. Con, seedlings without any treatment.

FIG. 6 illustrates expression of ABA- and stress-responsive genes in abo1-2 (M) and wild-type (W) seedlings treated with 100 μM ABA for 5 h (ABA-5). A tubulin gene was used as a loading control. Con, seedlings without any treatment.

FIG. 7 illustrates comparison of stomatal morphologies of abo1-1 and wild-type plants. (A) Light microscopy of abaxial epidermises from mature wild-type (a) and abo1-1 (b) leaves. In wild-type plants, only normally developed guard cells with formal stomata are observed. In contrast, in the abo1-1 mutant, only one pair of guard cells forms normal stoma among two pairs of adjacent stomata. Bar, 10 μm. (B) Scanning electron microscopy of abo1-1 (abnormal) (a, b, and c) and wild-type (d) stomata. (C and D) Quantitative analyses of (C) the numbers of stomata and (D) the pairs of guard cells in abaxial epidermises of abo1-1 and wild-type (WT) plants. Values are means ±SE (n=30). **, P<0.01.

FIG. 8 illustrates positional cloning and expression pattern of the ABO1 gene. (A) Positional cloning of the ABO1 gene. Chr, chromosome; BAC, bacterial artificial chromosome. (B) Structure of the ABO1 gene, showing the different mutant alleles. LB, T-DNA left border. (C) Expression of the ABO1 gene in different mutant alleles. An rRNA gene was used as a loading control. WT, wild type. (D) ABO1 expression was not induced under different treatment conditions. A tubulin gene was used as a control. Con, seedlings without any treatment; SA, salicylic acid; JA, jasmonic acid. (E) ABO1 promoter-GUS analysis in Arabidopsis transgenic seedlings. (a) One-day-old seedling, (b) 4-day-old seedling, (c) stem, (d) 10-day-old seedling, (e) siliques, and (f) flower. (F) Guard cells on an abaxial epidermis from a nontransgenic plant, used as a negative control. (G) ABO1 promoter-GUS analysis of guard cells on an abaxial epidermis from a transgenic plant. Bar, 10 μm.

FIG. 9 illustrates cross-complementation studies of a yeast tot1/elp1[Delta] Elongator mutant by expression of plant ABO1/ELO2. (A) Caffeine sensitivity. LFY3 (tot1/elp1[Delta]) cells transformed with vector pFF14 (TOT1/ELP1) or pDJ98 (ABO1/ELO2) or empty vector (2 μm) were serially diluted and spotted onto ABO1/ELO2-repressing (glucose [glc]) or -inducing (galactose [gal]) YPD medium in the absence (−caffeine) or presence (+caffeine) of 7.5 mM caffeine. Growth proceeded for 2 to 3 days at 30° C. Caffeine-sensitive (CafS) and -resistant (CafR) drug responses are indicated. (B) Thermosensitivity. LFY3 (tot1/elp1[Delta]) transformants (transformed as described for panel A) were replica plated on ABO1/ELO2-inducing galactose YPD medium and grown for 3 days at 30° C., 37° C., and 39° C. Thermotolerant (Ts+) responses are distinguished from sensitive (Ts−) and hypersensitive (Ts−−) responses. (C) Resistance to zymocin γ-toxin (γ-tox) subunit. LFY3 (tot1/elp1[Delta]) cells (transformed as described for panel A) were transformed with GAL1-γ-toxin expression vector (pHMS14) (top panels) or empty GAL1 control (pHMS22) (bottom panels). Upon replica spotting from glucose-repressing to galactose-inducing synthetic complete medium, cell death by γ-toxin was monitored (γ-tox: on). pHMS22 transformants carrying no γ-toxin (−γ-tox) served as galactose-utilizing (Gal+) controls. Growth on galactose in the presence of γ-toxin (+γ-tox) reflects resistance towards γ-toxin (ToxR), and failure to do so equals sensitivity (ToxS).

FIG. 10 illustrates that ABO1 negatively affects expression of ABAR (also known as GUN5).

### DETAILED DESCRIPTION

### I. Introduction

The present inventors have identified a mutant, abo1, in Arabidopsis, with a heightened sensitivity to abscisic acid and having a drought-resistance phenotype. The genetic locus responsible for this phenotype is abo1, a new allele of ELO2, which encodes a homolog of yeast Iki3/Elp1/Tot1 or human IκB kinase-associated protein (IKAP). Yeast Iki3 is the largest subunit of Elongator, a complex previously known to play a role in a number of physiological processes including mRNA transcription enlongation.

Abscisic acid is a multifunctional phytohormone involved in a variety of important protective functions including bud dormancy, seed dormancy and/or maturation, abscission of leaves and fruits, and response to a wide variety of biological stresses (e.g. cold, heat, salinity, and drought). ABA is also responsible for regulating stomatal closure by a mechanism independent of CO₂ concentration. Thus, because it has been surprisingly found that inhibition of ELO2 results in increases ABA sensitivity, these phenotypes can be modulated by modulating expression of ELO2 or orthologs of ELO2 in other plants. Phenotypes that are induced by ABA can be increased or speeded in plants with decreased expression of ELO2 or orthologs thereof, whereas it is believed that such phenotypes can be reduced or slowed in plants with increased expression of ELO2 or orthologs thereof. ELO2 mediates ABA signaling as a negative regulator in, for example, seed germination, post-germination growth, stomatal movement and plant tolerance to stress including, but not limited to, drought. Accordingly, when abscisic acid sensitivity is increased by reducing expression of ELO2 or orthologs thereof, desirable characteristics in plants such as increased stress (e.g., drought) tolerance and delayed seed germination is achieved.

This invention therefore provides a new method for generating transgenic plants with enhanced drought-resistance, by inhibiting or reducing the endogenous wild-type ELO2 gene expression or activity in the plants. The inhibition or reduction may be achieved, for example, at the transcription level by antisense, sense suppression or RNAi techniques; or it may be achieved at the protein activity level by introducing into the plants a dominant negative ABO1 protein.

### II. ELO2

Several ELO2 polypeptide sequences (i.e., ELO2 or orthologs thereof) are known in the art and can be used according to the methods and compositions of the invention. A listing of some known ELO2 polypeptide and polynucleotide sequences from various species is provided in Table 1.

TABLE 1

| Plant species | Protein | Coding or cDNA sequence |
|---|---|---|
| Arabidopsis | SEQ ID NO: 2 | SEQ ID NO: 1 |
| Rice | SEQ ID NO: 4 | SEQ ID NO: 3 |
| Grape | SEQ ID NO: 6 | SEQ ID NO: 5 |

The present invention provides for use of the above proteins and/or nucleic acid sequences, or sequences substantially identical (e.g., 50%, 60%, 70%, 75%, 80%, 85%, 90%, 95%, 98% identical) to those listed above in the methods and compositions (e.g., expression cassettes, plants, etc.) of the present invention. In situations where variants of the above sequences are desired, it can be useful to generate sequence alignments to identify conserved amino acid or motifs (i.e., where alteration in sequences may alter protein function) and regions where variation occurs in alignment of sequences (i.e., where variation of sequence is not likely to significantly affect protein activity).

As can be seen in Table 2 below, a number of known plant ELO2 polypeptides are at least about 50% identical to the Arabidopsis sequence (SEQ ID NO:2).

TABLE 2

| | | Percent |
|---|---|---|
| Organism | Aligned to | Identity |
| SEQ ID NO: 2 | SEQ ID NO: 2 | 100% |
| Arabidopsis | Arabidopsis | |
| thaliana sequence | thaliana sequence | |
| Genbank | Genbank | |
| #NP_196872.1 | #NP_196872.1 | |
| SEQ ID NO: 4 | SEQ ID NO: 2 | 50.2% |
| Oryza | Arabidopsis | |
| sativa sequence | thaliana sequence | |
| Genbank | Genbank | |
| #NP_001059998.1 | #NP_196872.1 | |
| SEQ ID NO: 6 | SEQ ID NO: 2 | 50.3% |
| Vitis | Arabidopsis | |
| vinifera | thaliana sequence | |
| Genbank | Genbank | |
| #CAN82069.1 | #NP_196872.1 | |

The isolation of a polynucleotide sequence encoding a plant ELO2 (e.g., from plants where ELO2 sequences have not yet been identified) may be accomplished by a number of techniques. For instance, oligonucleotide probes based on the ELO2 coding sequences disclosed (e.g., as listed in Table 1) here can be used to identify the desired ELO2 gene in a cDNA or genomic DNA library. To construct genomic libraries, large segments of genomic DNA are generated by random fragmentation, e.g., using restriction endonucleases, and are ligated with vector DNA to form concatemers that can be packaged into the appropriate vector. To prepare a cDNA library, mRNA is isolated from the desired tissue, such as a leaf from a particular plant species, and a cDNA library containing the gene transcript of interest is prepared from the mRNA. Alternatively, cDNA may be prepared from mRNA extracted from other tissues in which ELO2 gene is expressed.

The cDNA or genomic library can then be screened using a probe based upon the sequence of a ELO2 gene disclosed here (e.g., as listed in Table 1). Probes may be used to hybridize with genomic DNA or cDNA sequences to isolate homologous genes in the same or different plant species. Alternatively, antibodies raised against a polypeptide can be used to screen an mRNA expression library.

Alternatively, the nucleic acids encoding ELO2 can be amplified from nucleic acid samples using amplification techniques. For instance, polymerase chain reaction (PCR) technology can be used to amplify the coding sequences of ELO2 directly from genomic DNA, from cDNA, from genomic libraries or cDNA libraries. PCR and other in vitro amplification methods may also be useful, for example, to clone polynucleotide sequences encoding ELO2 to be expressed, to make nucleic acids to use as probes for detecting the presence of the desired mRNA in samples, for nucleic acid sequencing, or for other purposes. For a general overview of PCR see PCR Protocols: A Guide to Methods and Applications. (Innis, M, Gelfand, D., Sninsky, J. and White, T., eds.), Academic Press, San Diego (1990). Appropriate primers and probes for identifying sequences from plant tissues are generated from comparisons of the sequences provided here with other related genes.

The advancement in studies of plant genomes also permits a person of skill in the art to quickly determine the ELO2 coding sequence for a selected plant. For instance, the partial or entire genome of a number of plants has been sequenced and open reading frames identified. By a routine BLAST search, one can immediately identify the coding sequence for ELO2 in various plants.

### III. Use of ELO2 Nucleic Acids of the Invention

The invention provides methods of modulating ABA sensitivity in a plant by altering ELO2 expression or activity, for example, by introducing into a plant a recombinant expression cassette comprising a regulatory element (e.g., a promoter) operably linked to a ELO2 polynucleotide, i.e., a nucleic acid encoding ELO2 or a sequence comprising a portion of the sequence of an ELO2 cDNA or complement thereof.

In some embodiments, the methods of the invention comprise decreasing endogenous ELO2 expression in plant, thereby increasing ABA sensitivity in the plant. Such methods can involve, for example, mutagenesis (e.g., chemical, radiation, transposon or other mutagenesis) of ELO2 sequences in a plant to reduce ELO2 expression or activity (and subsequent selection of plants with mutated ELO2 sequences and/or increased ABA sensitivity), or introduction of a polynucleotide substantially identical to at least a portion of a ELO2 cDNA sequence or a complement thereof (e.g., an “RNAi construct”) to reduce ELO2 expression (and subsequent selection of plants with mutated ELO2 sequences and/or increased ABA sensitivity). Decreased ELO2 expression is useful for increasing ABA sensitivity of a plant, and resulting in, for example, improved stress (e.g., drought) tolerance and/or delayed seed germination (to avoid pre-mature germination, for example as can occur in humid environments or due to other exposure to moisture). For stress tolerance, promoters can be selected that are generally constitutive and are expressed in most plant tissues, or can be leaf or root specific. To affect seed germination, promoters are generally used that result in expression in seed or, in some embodiments, floral organs or embryos.

A number of methods can be used to inhibit gene expression in plants. For instance, antisense technology can be conveniently used. To accomplish this, a nucleic acid segment from the desired gene is cloned and operably linked to a promoter such that the antisense strand of RNA will be transcribed. The expression cassette is then transformed into plants and the antisense strand of RNA is produced. In plant cells, it has been suggested that antisense RNA inhibits gene expression by preventing the accumulation of mRNA which encodes the enzyme of interest, see, e.g., Sheehy et al., Proc. Nat. Acad. Sci. USA, 85:8805-8809 (1988); Pnueli et al., The Plant Cell 6:175-186 (1994); and Hiatt et al., U.S. Pat. No. 4,801,340.

The antisense nucleic acid sequence transformed into plants will be substantially identical to at least a portion of the endogenous gene or genes to be repressed. The sequence, however, does not have to be perfectly identical to inhibit expression. Thus, an antisense or sense nucleic acid molecule encoding only a portion of ELO2, or a portion of the ELO2 cDNA, can be useful for producing a plant in which ELO2 expression is suppressed. The vectors of the present invention can be designed such that the inhibitory effect applies to other proteins within a family of genes exhibiting homology or substantial homology to the target gene.

For antisense suppression, the introduced sequence also need not be full length relative to either the primary transcription product or fully processed mRNA. Generally, higher homology can be used to compensate for the use of a shorter sequence. Furthermore, the introduced sequence need not have the same intron or exon pattern, and homology of non-coding segments may be equally effective. For example, a sequence of between about 30 or 40 nucleotides can be used, and in some embodiments, about full length nucleotides should be used, though a sequence of, for example, at least about 20, 50 100, 200, or 500 nucleotides substantially identical to SEQ ID NO: 1, 3 or 5, or an endogenous ELO2 mRNA or cDNA can be used.

Catalytic RNA molecules or ribozymes can also be used to inhibit expression of ELO2 genes. It is possible to design ribozymes that specifically pair with virtually any target RNA and cleave the phosphodiester backbone at a specific location, thereby functionally inactivating the target RNA. In carrying out this cleavage, the ribozyme is not itself altered, and is thus capable of recycling and cleaving other molecules, making it a true enzyme. The inclusion of ribozyme sequences within antisense RNAs confers RNA-cleaving activity upon them, thereby increasing the activity of the constructs.

A number of classes of ribozymes have been identified. One class of ribozymes is derived from a number of small circular RNAs that are capable of self-cleavage and replication in plants. The RNAs replicate either alone (viroid RNAs) or with a helper virus (satellite RNAs). Examples include RNAs from avocado sunblotch viroid and the satellite RNAs from tobacco ringspot virus, lucerne transient streak virus, velvet tobacco mottle virus, solanum nodiflorum mottle virus and subterranean clover mottle virus. The design and use of target RNA-specific ribozymes is described in Haseloff et al. Nature, 334:585-591 (1988).

Another method of suppression is sense suppression (also known as co-suppression). Introduction of expression cassettes in which a nucleic acid is configured in the sense orientation with respect to the promoter has been shown to be an effective means by which to block the transcription of target genes. For an example of the use of this method to modulate expression of endogenous genes see, Napoli et al., The Plant Cell 2:279-289 (1990); Flavell, Proc. Natl. Acad. Sci., USA 91:3490-3496 (1994); Kooter and Mol, Current Opin. Biol. 4:166-171 (1993); and U.S. Pat. Nos. 5,034,323, 5,231,020, and 5,283,184.

Generally, where inhibition of expression is desired, some transcription of the introduced sequence occurs. The effect may occur where the introduced sequence contains no coding sequence per se, but only intron or untranslated sequences homologous to sequences present in the primary transcript of the endogenous sequence. The introduced sequence generally will be substantially identical to the endogenous sequence intended to be repressed. This minimal identity will typically be greater than about 65%, but a higher identity can exert a more effective repression of expression of the endogenous sequences. In some embodiments, sequences with substantially greater identity are used, e.g., at least about 80, at least about 95%, or 100% identity are used. As with antisense regulation, the effect can be designed and tested to apply to any other proteins within a similar family of genes exhibiting homology or substantial homology.

For sense suppression, the introduced sequence in the expression cassette, needing less than absolute identity, also need not be full length, relative to either the primary transcription product or fully processed mRNA. This may be preferred to avoid concurrent production of some plants that are overexpressers. A higher identity in a shorter than full length sequence compensates for a longer, less identical sequence. Furthermore, the introduced sequence need not have the same intron or exon pattern, and identity of non-coding segments will be equally effective. Normally, a sequence of the size ranges noted above for antisense regulation is used, i.e., 30-40, or at least about 20, 50, 100, 200, 500 or more nucleotides substantially identical to SEQ ID NO: 1, 3 or 5, or an endogenous ELO2 mRNA or cDNA.

Endogenous gene expression may also be suppressed by means of RNA interference (RNAi) (and indeed co-suppression can be considered a type of RNAi), which uses a double-stranded RNA having a sequence identical or similar to the sequence of the target gene. RNAi is the phenomenon in which when a double-stranded RNA having a sequence identical or similar to that of the target gene is introduced into a cell, the expressions of both the inserted exogenous gene and target endogenous gene are suppressed. The double-stranded RNA may be formed from two separate complementary RNAs or may be a single RNA with internally complementary sequences that form a double-stranded RNA. Although complete details of the mechanism of RNAi are still unknown, it is considered that the introduced double-stranded RNA is initially cleaved into small fragments, which then serve as indexes of the target gene in some manner, thereby degrading the target gene. RNAi is known to be also effective in plants (see, e.g., Chuang, C. F. & Meyerowitz, E. M., Proc. Natl. Acad. Sci. USA 97: 4985 (2000); Waterhouse et al., Proc. Natl. Acad. Sci. USA 95:13959-13964 (1998); Tabara et al. Science 282:430-431 (1998); Matthew, Comp Funct Genom 5: 240-244 (2004); Lu, et al., Nucleic Acids Research 32(21):e171 (2004)). For example, to achieve suppression of the expression of a DNA encoding a protein using RNAi, a double-stranded RNA (e.g., from promoters expressing both sense and complementary antisense sequences) having the sequence of a DNA encoding the protein, or a substantially similar sequence thereof (including those engineered not to translate the protein) or fragment thereof, is introduced into a plant of interest. The resulting plants may then be screened for a phenotype associated (e.g., increased ABA sensitivity and/or drought tolerance or other ABA phenotypes) with the target protein and/or by monitoring steady-state RNA levels for transcripts encoding the protein. The genes used for RNAi need not be completely identical to the target gene; they may be at least 70%, 80%, 90%, 95% or more identical to the target gene sequence or at least a contiguous sequence therefrom of at least 20, 50, 100, 200, or 500 nucleotides. See, e.g., U.S. Patent Publication No. 2004/0029283. The constructs encoding an RNA molecule with a stem-loop structure that is unrelated to the target gene and that is positioned distally to a sequence specific for the gene of interest may also be used to inhibit target gene expression. See, e.g., U.S. Patent Publication No. 2003/0221211.

The RNAi polynucleotides can encompass the full-length target RNA or may correspond to a fragment of the target RNA. In some cases, the fragment will have fewer than 100, 200, 300, 400, 500 600, 700, 800, 900 or 1,000 nucleotides corresponding to the target sequence. In addition, in some embodiments, these fragments are at least, e.g., 20, 50, 100, 150, 200, or more nucleotides in length. In some cases, fragments for use in RNAi will be at least substantially similar to regions of a target protein that do not occur in other proteins in the organism or may be selected to have as little similarity to other organism transcripts as possible, e.g., selected by comparison to sequences in analyzing publicly-available sequence databases.

Expression vectors that continually express siRNA in transiently- and stably-transfected have been engineered to express small hairpin RNAs, which get processed in vivo into siRNAs molecules capable of carrying out gene-specific silencing (Brummelkamp et al., Science 296:550-553 (2002), and Paddison, et al., Genes & Dev. 16:948-958 (2002)). Post-transcriptional gene silencing by double-stranded RNA is discussed in further detail by Hammond et al. Nature Rev Gen 2: 110-119 (2001), Fire et al. Nature 391: 806-811 (1998) and Timmons and Fire Nature 395: 854 (1998).

One of skill in the art will recognize that using technology based on specific nucleotide sequences (e.g., antisense or sense suppression technology), families of homologous genes can be suppressed with a single sense or antisense transcript. For instance, if a sense or antisense transcript is designed to have a sequence that is conserved among a family of genes, then multiple members of a gene family can be suppressed. Conversely, if the goal is to only suppress one member of a homologous gene family, then the sense or antisense transcript should be targeted to sequences with the most variance between family members.

Another means of inhibiting ELO2 function in a plant is by creation of dominant negative mutations. In this approach, non-functional, mutant ELO2 polypeptides, which retain the ability to interact with wild-type subunits are introduced into a plant. A dominant negative construct also can be used to suppress ELO2 expression in a plant. A dominant negative construct useful in the invention generally contains a portion of the complete ELO2 coding sequence sufficient, for example, for DNA-binding or for a protein-protein interaction such as a homodimeric or heterodimeric protein-protein interaction but lacking the transcriptional activity of the wild type protein.

### IV. Recombinant Expression Vector

Once the coding or cDNA sequence for ELO2 is obtained, it can also be used to prepare an expression cassette for expressing the ELO2 protein in a transgenic plant, directed by a heterologous promoter. Alternatively, as described above, expression vectors can be used to express ELO2 polynucleotides and variants thereof that inhibit endogenous ELO2 expression.

Any of a number of means well known in the art can be used to increase or decrease ELO2 activity or expression in plants. Any organ can be targeted, such as shoot vegetative organs/structures (e.g. leaves, stems and tubers), roots, flowers and floral organs/structures (e.g. bracts, sepals, petals, stamens, carpels, anthers and ovules), seed (including embryo, endosperm, and seed coat) and fruit. Alternatively, the ELO2 gene or fragments or variants thereof can be expressed constitutively (e.g., using the CaMV 35S promoter).

To use ELO2 coding or cDNA sequences in the above techniques, recombinant DNA vectors suitable for transformation of plant cells are prepared. Techniques for transforming a wide variety of higher plant species are well known and described in the technical and scientific literature. See, e.g., Weising et al. Ann. Rev. Genet. 22:421-477 (1988). A DNA sequence coding for the ELO2 polypeptide preferably will be combined with transcriptional and translational initiation regulatory sequences which will direct the transcription of the sequence from the gene in the intended tissues of the transformed plant.

For example, a plant promoter fragment may be employed to direct expression of the ELO2 gene or fragments or variants thereof in all tissues of a regenerated plant. Such promoters are referred to herein as “constitutive” promoters and are active under most environmental conditions and states of development or cell differentiation. Examples of constitutive promoters include the cauliflower mosaic virus (CaMV) 35S transcription initiation region, the 1′- or 2′-promoter derived from T-DNA of Agrobacterium tumafaciens, and other transcription initiation regions from various plant genes known to those of skill.

Alternatively, the plant promoter may direct expression of the ELO2 inhibiting sequences (e.g., antisense or RNAi constructs) in a specific tissue (tissue-specific promoters) or may be otherwise under more precise environmental control (inducible promoters). Examples of tissue-specific promoters under developmental control include promoters that initiate transcription only in certain tissues, such as leaves or guard cells (including but not limited to those described in WO/2005/085449; U.S. Pat. No. 6,653,535; Li et al., Sci China C Life Sci. 2005 April; 48(2):181-6; Husebye, et al., Plant Physiol, April 2002, Vol. 128, pp. 1180-1188; and Plesch, et al., Gene, Volume 249, Number 1, 16 May 2000, pp. 83-89(7)). Examples of environmental conditions that may affect transcription by inducible promoters include anaerobic conditions, elevated temperature, or the presence of light.

If proper protein expression is desired, a polyadenylation region at the 3′-end of the coding region should be included. The polyadenylation region can be derived from the natural gene, from a variety of other plant genes, or from T-DNA.

The vector comprising the sequences (e.g., promoters or ELO2 coding regions) will typically comprise a marker gene that confers a selectable phenotype on plant cells. For example, the marker may encode biocide resistance, particularly antibiotic resistance, such as resistance to kanamycin, G418, bleomycin, hygromycin, or herbicide resistance, such as resistance to chlorosluforon or Basta.

The invention provides a ELO2 nucleic acid operably linked to a promoter which, in some embodiments, is capable of driving the transcription of the ELO2 cDNA or coding sequence or fragments or variants thereof in plants. The promoter can be, e.g., derived from plant or viral sources. The promoter can be, e.g., constitutively active, inducible, or tissue specific. In construction of recombinant expression cassettes, vectors, transgenics, of the invention, a different promoters can be chosen and employed to differentially direct gene expression, e.g., in some or all tissues of a plant or animal.

A. Constitutive Promoters

A promoter fragment can be employed to direct expression of a ELO2 nucleic acid in all transformed cells or tissues, e.g., as those of a regenerated plant. The term “constitutive regulatory element” means a regulatory element that confers a level of expression upon an operatively linked nucleic molecule that is relatively independent of the cell or tissue type in which the constitutive regulatory element is expressed. A constitutive regulatory element that is expressed in a plant generally is widely expressed in a large number of cell and tissue types. Promoters that drive expression continuously under physiological conditions are referred to as “constitutive” promoters and are active under most environmental conditions and states of development or cell differentiation.

A variety of constitutive regulatory elements useful for ectopic expression in a transgenic plant are well known in the art. The cauliflower mosaic virus 35S (CaMV 35S) promoter, for example, is a well-characterized constitutive regulatory element that produces a high level of expression in all plant tissues (Odell et al., Nature 313:810-812 (1985)). The CaMV 35S promoter can be particularly useful due to its activity in numerous diverse plant species (Benfey and Chua, Science 250:959-966 (1990); Futterer et al., Physiol. Plant 79:154 (1990); Odell et al., supra, 1985). A tandem 35S promoter, in which the intrinsic promoter element has been duplicated, confers higher expression levels in comparison to the unmodified 35S promoter (Kay et al., Science 236:1299 (1987)). Other useful constitutive regulatory elements include, for example, the cauliflower mosaic virus 19S promoter; the Figwort mosaic virus promoter; and the nopaline synthase (nos) gene promoter (Singer et al., Plant Mol. Biol. 14:433 (1990); An, Plant Physiol. 81:86 (1986)).

Additional constitutive regulatory elements including those for efficient expression in monocots also are known in the art, for example, the pEmu promoter and promoters based on the rice Actin-1 5′ region (Last et al., Theor. Appl. Genet. 81:581 (1991); Mcelroy et al., Mol. Gen. Genet. 231:150 (1991); Mcelroy et al., Plant Cell 2:163 (1990)). Chimeric regulatory elements, which combine elements from different genes, also can be useful for ectopically expressing a nucleic acid molecule encoding a ELO2 protein (Comai et al., Plant Mol. Biol. 15:373 (1990)).

Other examples of constitutive promoters include the 1′- or 2′-promoter derived from T-DNA of Agrobacterium tumefaciens (see, e.g., Mengiste (1997) supra; O'Grady (1995) Plant Mol. Biol. 29:99-108); actin promoters, such as the Arabidopsis actin gene promoter (see, e.g., Huang (1997) Plant Mol. Biol. 1997 33:125-139); alcohol dehydrogenase (Adh) gene promoters (see, e.g., Millar (1996) Plant Mol. Biol. 31:897-904); ACT11 from Arabidopsis (Huang et al. Plant Mol. Biol. 33:125-139 (1996)), Cat3 from Arabidopsis (GenBank No. U43147, Zhong et al., Mol. Gen. Genet. 251:196-203 (1996)), the gene encoding stearoyl-acyl carrier protein desaturase from Brassica napus (Genbank No. X74782, Solocombe et al. Plant Physiol. 104:1167-1176 (1994)), GPc1 from maize (GenBank No. X15596, Martinez et al. J. Mol. Biol. 208:551-565 (1989)), Gpc2 from maize (GenBank No. U45855, Manjunath et al., Plant Mol. Biol. 33:97-112 (1997)), other transcription initiation regions from various plant genes known to those of skill. See also Holtorf Plant Mol. Biol. 29:637-646 (1995).

B. Inducible Promoters

Alternatively, a plant promoter may direct expression of the ELO2 gene under the influence of changing environmental conditions or developmental conditions. Examples of environmental conditions that may effect transcription by inducible promoters include anaerobic conditions, elevated temperature, drought, or the presence of light. Such promoters are referred to herein as “inducible” promoters. For example, the invention can incorporate drought-specific promoter such as, but not limited to, the drought-inducible promoter of maize (Busk (1997) supra); or alternatively the cold, drought, and high salt inducible promoter from potato (Kirch (1997) Plant Mol. Biol. 33:897-909).

Alternatively, plant promoters which are inducible upon exposure to plant hormones, such as auxins, are used to express the ELO2 gene or fragments or variants thereof. For example, the invention can use the auxin-response elements E1 promoter fragment (AuxREs) in the soybean (Glycine max L.) (Liu (1997) Plant Physiol. 115:397-407); the auxin-responsive Arabidopsis GST6 promoter (also responsive to salicylic acid and hydrogen peroxide) (Chen (1996) Plant J. 10: 955-966); the auxin-inducible parC promoter from tobacco (Sakai (1996) 37:906-913); a plant biotin response element (Streit (1997) Mol. Plant. Microbe Interact. 10:933-937); and, the promoter responsive to the stress hormone abscisic acid (Sheen (1996) Science 274:1900-1902).

Plant promoters inducible upon exposure to chemicals reagents that may be applied to the plant, such as herbicides or antibiotics, are also useful for expressing the ELO2 gene or fragments or variants thereof. For example, the maize In2-2 promoter, activated by benzenesulfonamide herbicide safeners, can be used (De Veylder (1997) Plant Cell Physiol. 38:568-577); application of different herbicide safeners induces distinct gene expression patterns, including expression in the root, hydathodes, and the shoot apical meristem. A ELO2 coding sequence can also be under the control of, e.g., a tetracycline-inducible promoter, e.g., as described with transgenic tobacco plants containing the Avena sativa L. (oat) arginine decarboxylase gene (Masgrau (1997) Plant J. 11:465-473); or, a salicylic acid-responsive element (Stange (1997) Plant J. 11:1315-1324; Uknes et al., Plant Cell 5:159-169 (1993); Bi et al., Plant J. 8:235-245 (1995)).

Examples of useful inducible regulatory elements include copper-inducible regulatory elements (Mett et al., Proc. Natl. Acad. Sci. USA 90:4567-4571 (1993); Furst et al., Cell 55:705-717 (1988)); tetracycline and chlor-tetracycline-inducible regulatory elements (Gatz et al., Plant J. 2:397-404 (1992); Röder et al., Mol. Gen. Genet. 243:32-38 (1994); Gatz, Meth. Cell Biol. 50:411-424 (1995)); ecdysone inducible regulatory elements (Christopherson et al., Proc. Natl. Acad. Sci. USA 89:6314-6318 (1992); Kreutzweiser et al., Ecotoxicol. Environ. Safety 28:14-24 (1994)); heat shock inducible regulatory elements (Takahashi et al., Plant Physiol. 99:383-390 (1992); Yabe et al., Plant Cell Physiol. 35:1207-1219 (1994); Ueda et al., Mol. Gen. Genet. 250:533-539 (1996)); and lac operon elements, which are used in combination with a constitutively expressed lac repressor to confer, for example, IPTG-inducible expression (Wilde et al., EMBO J. 11:1251-1259 (1992)). An inducible regulatory element useful in the transgenic plants of the invention also can be, for example, a nitrate-inducible promoter derived from the spinach nitrite reductase gene (Back et al., Plant Mol. Biol. 17:9 (1991)) or a light-inducible promoter, such as that associated with the small subunit of RuBP carboxylase or the LHCP gene families (Feinbaum et al., Mol. Gen. Genet. 226:449 (1991); Lam and Chua, Science 248:471 (1990)).

C. Tissue-Specific Promoters

Alternatively, the plant promoter may direct expression of the ELO2 gene or fragments or variants thereof in a specific tissue (tissue-specific promoters). Tissue specific promoters are transcriptional control elements that are only active in particular cells or tissues at specific times during plant development, such as in vegetative tissues or reproductive tissues.

Examples of tissue-specific promoters under developmental control include promoters that initiate transcription only (or primarily only) in certain tissues, such as vegetative tissues, e.g., roots or leaves, or reproductive tissues, such as fruit, ovules, seeds, pollen, pistols, flowers, or any embryonic tissue. Reproductive tissue-specific promoters may be, e.g., ovule-specific, embryo-specific, endosperm-specific, integument-specific, seed and seed coat-specific, pollen-specific, petal-specific, sepal-specific, or some combination thereof.

Other tissue-specific promoters include seed promoters. Suitable seed-specific promoters are derived from the following genes: MAC1 from maize (Sheridan (1996) Genetics 142:1009-1020); Cat3 from maize (GenBank No. L05934, Abler (1993) Plant Mol. Biol. 22:10131-1038); vivparous-1 from Arabidopsis (Genbank No. U93215); atmycl from Arabidopsis (Urao (1996) Plant Mol. Biol. 32:571-57; Conceicao (1994) Plant 5:493-505); napA from Brassica napus (GenBank No. J02798, Josefsson (1987) JBL 26:12196-1301); and the napin gene family from Brassica napus (Sjodahl (1995) Planta 197:264-271).

A variety of promoters specifically active in vegetative tissues, such as leaves, stems, roots and tubers, can also be used to express the ELO2 gene or fragments or variants thereof. For example, promoters controlling patatin, the major storage protein of the potato tuber, can be used, see, e.g., Kim (1994) Plant Mol. Biol. 26:603-615; Martin (1997) Plant J. 11:53-62. The ORF13 promoter from Agrobacterium rhizogenes that exhibits high activity in roots can also be used (Hansen (1997) Mol. Gen. Genet. 254:337-343. Other useful vegetative tissue-specific promoters include: the tarin promoter of the gene encoding a globulin from a major taro (Colocasia esculenta L. Schott) corm protein family, tarin (Bezerra (1995) Plant Mol. Biol. 28:137-144); the curculin promoter active during taro corm development (de Castro (1992) Plant Cell 4:1549-1559) and the promoter for the tobacco root-specific gene TobRB7, whose expression is localized to root meristem and immature central cylinder regions (Yamamoto (1991) Plant Cell 3:371-382).

Leaf-specific promoters, such as the ribulose biphosphate carboxylase (RBCS) promoters can be used. For example, the tomato RBCS1, RBCS2 and RBCS3A genes are expressed in leaves and light-grown seedlings, only RBCS1 and RBCS2 are expressed in developing tomato fruits (Meier (1997) FEBS Lett. 415:91-95). A ribulose bisphosphate carboxylase promoters expressed almost exclusively in mesophyll cells in leaf blades and leaf sheaths at high levels, described by Matsuoka (1994) Plant J. 6:311-319, can be used. Another leaf-specific promoter is the light harvesting chlorophyll a/b binding protein gene promoter, see, e.g., Shiina (1997) Plant Physiol. 115:477-483; Casal (1998) Plant Physiol. 116:1533-1538. The Arabidopsis thaliana myb-related gene promoter (Atmyb5) described by Li (1996) FEBS Lett. 379:117-121, is leaf-specific. The Atmyb5 promoter is expressed in developing leaf trichomes, stipules, and epidermal cells on the margins of young rosette and cauline leaves, and in immature seeds. Atmyb5 mRNA appears between fertilization and the 16 cell stage of embryo development and persists beyond the heart stage. A leaf promoter identified in maize by Busk (1997) Plant J. 11:1285-1295, can also be used.

Another class of useful vegetative tissue-specific promoters are meristematic (root tip and shoot apex) promoters. For example, the “SHOOTMERISTEMLESS” and “SCARECROW” promoters, which are active in the developing shoot or root apical meristems, described by Di Laurenzio (1996) Cell 86:423-433; and, Long (1996) Nature 379:66-69; can be used. Another useful promoter is that which controls the expression of 3-hydroxy-3-methylglutaryl coenzyme A reductase HMG2 gene, whose expression is restricted to meristematic and floral (secretory zone of the stigma, mature pollen grains, gynoecium vascular tissue, and fertilized ovules) tissues (see, e.g., Enjuto (1995) Plant Cell. 7:517-527). Also useful are kn1-related genes from maize and other species which show meristem-specific expression, see, e.g., Granger (1996) Plant Mol. Biol. 31:373-378; Kerstetter (1994) Plant Cell 6:1877-1887; Hake (1995) Philos. Trans. R. Soc. Lond. B. Biol. Sci. 350:45-51. For example, the Arabidopsis thaliana KNAT1 promoter (see, e.g., Lincoln (1994) Plant Cell 6:1859-1876).

One of skill will recognize that a tissue-specific promoter may drive expression of operably linked sequences in tissues other than the target tissue. Thus, as used herein a tissue-specific promoter is one that drives expression preferentially in the target tissue, but may also lead to some expression in other tissues as well.

In another embodiment, the ELO2 gene or fragments or variants thereof is expressed through a transposable element. This allows for constitutive, yet periodic and infrequent expression of the constitutively active polypeptide. The invention also provides for use of tissue-specific promoters derived from viruses including, e.g., the tobamovirus subgenomic promoter (Kumagai (1995) Proc. Natl. Acad. Sci. USA 92:1679-1683; the rice tungro bacilliform virus (RTBV), which replicates only in phloem cells in infected rice plants, with its promoter which drives strong phloem-specific reporter gene expression; the cassava vein mosaic virus (CVMV) promoter, with highest activity in vascular elements, in leaf mesophyll cells, and in root tips (Verdaguer (1996) Plant Mol. Biol. 31:1129-1139).

### V. Production of Transgenic Plants

As detailed herein, the present invention provides for transgenic plants comprising recombinant expression cassettes either for expressing polynucleotides to inhibit expression of endogenous ELO2 in a plant. Thus, in some embodiments, a transgenic plant is generated that contains a complete or partial sequence of an endogenous ELO2 encoding polynucleotide, for reducing ELO2 expression and activity. In some embodiments, a transgenic plant is generated that contains a complete or partial sequence of a polynucleotide that is substantially identical to an endogenous ELO2 encoding polynucleotide, reducing ELO2 expression and activity. In some embodiments, a transgenic plant is generated that contains a complete or partial sequence of a polynucleotide that is from a species other than the species of the transgenic plant. It should be recognized that transgenic plants encompass the plant or plant cell in which the expression cassette is introduced as well as progeny of such plants or plant cells that contain the expression cassette, including the progeny that have the expression cassette stably integrated in a chromosome.

A recombinant expression vector comprising a ELO2 coding sequence, cDNA, or fragment or variant thereof, driven by a heterologous promoter, may be introduced into the genome of the desired plant host by a variety of conventional techniques. For example, the DNA construct may be introduced directly into the genomic DNA of the plant cell using techniques such as electroporation and microinjection of plant cell protoplasts, or the DNA construct can be introduced directly to plant tissue using ballistic methods, such as DNA particle bombardment. Alternatively, the DNA construct may be combined with suitable T-DNA flanking regions and introduced into a conventional Agrobacterium tumefaciens host vector. The virulence functions of the Agrobacterium tumefaciens host will direct the insertion of the construct and adjacent marker into the plant cell DNA when the cell is infected by the bacteria. While transient expression of ELO2 is encompassed by the invention, in some embodiments, expression of construction of the invention will be from insertion of expression cassettes into the plant genome, e.g., such that at least some plant offspring also contain the integrated expression cassette.

Microinjection techniques are also useful for this purpose. These techniques are well known in the art and thoroughly described in the literature. The introduction of DNA constructs using polyethylene glycol precipitation is described in Paszkowski et al. Embo J. 3:2717-2722 (1984). Electroporation techniques are described in Fromm et al. Proc. Natl. Acad. Sci. USA 82:5824 (1985). Ballistic transformation techniques are described in Klein et al. Nature 327:70-73 (1987).

Agrobacterium tumefaciens-mediated transformation techniques, including disarming and use of binary vectors, are well described in the scientific literature. See, for example, Horsch et al. Science 233:496-498 (1984), and Fraley et al. Proc. Natl. Acad. Sci. USA 80:4803 (1983).

Transformed plant cells derived by any of the above transformation techniques can be cultured to regenerate a whole plant that possesses the transformed genotype and thus the desired phenotype such as enhanced drought-resistance. Such regeneration techniques rely on manipulation of certain phytohormones in a tissue culture growth medium, typically relying on a biocide and/or herbicide marker which has been introduced together with the desired nucleotide sequences. Plant regeneration from cultured protoplasts is described in Evans et al., Protoplasts Isolation and Culture, Handbook of Plant Cell Culture, pp. 124-176, MacMillilan Publishing Company, New York, 1983; and Binding, Regeneration of Plants, Plant Protoplasts, pp. 21-73, CRC Press, Boca Raton, 1985. Regeneration can also be obtained from plant callus, explants, organs, or parts thereof. Such regeneration techniques are described generally in Klee et al. Ann. Rev. of Plant Phys. 38:467-486 (1987).

One of skill will recognize that after the expression cassette is stably incorporated in transgenic plants and confirmed to be operable, it can be introduced into other plants by sexual crossing. Any of a number of standard breeding techniques can be used, depending upon the species to be crossed.

The expression cassettes of the invention can be used to confer drought resistance on essentially any plant. Thus, the invention has use over a broad range of plants, including species from the genera Asparagus, Atropa, Avena, Brassica, Citrus, Citrullus, Capsicum, Cucumis, Cucurbita, Daucus, Fragaria, Glycine, Gossypium, Helianthus, Heterocallis, Hordeum, Hyoscyamus, Lactuca, Linum, Lolium, Lycopersicon, Malus, Manihot, Majorana, Medicago, Nicotiana, Oryza, Panieum, Pannesetum, Persea, Pisum, Pyrus, Prunus, Raphanus, Secale, Senecio, Sinapis, Solanum, Sorghum, Trigonella, Triticum, Vitis, Vigna, and, Zea. In some embodiments, the plant is selected from the group consisting of rice, maize, wheat, soybeans, cotton, canola, and alfalfa. In some embodiments, the plant is an ornamental plant. In some embodiment, the plant is a vegetable- or fruit-producing plant.

In some embodiments, the methods of the invention are used to confer drought-resistance on turf grasses. A number of turf grasses are known to those of skill in the art. For example, fescue, Festuca spp. (e.g., F. arundinacea, F. rubra, F. ovina var. duriuscula, and F. ovina) can be used. Other grasses include Kentucky bluegrass Poa pratensis and creeping bentgrass Agrostis palustris.

Those of skill will recognize that a number of plant species can be used as models to predict the phenotypic effects of transgene expression in other plants. For example, it is well recognized that both tobacco (Nicotiana) and Arabidopsis plants are useful models of transgene expression, particularly in other dicots.

The plants of the invention have either enhanced or reduced abscisic acid sensitivity compared to plants otherwise genetically identical except for expression of ELO2. Abscisic acid sensitivity can be monitored by observing or measuring any phenotype mediated by ABA. Those of skill in the art will recognize that ABA is a well-studied plant hormone and that ABA mediates many changes in characteristics, any of which can be monitored to determined whether ABA sensitivity has been modulated. In some embodiments, modulated ABA sensitivity is manifested by altered timing of seed germination or altered stress (e.g., drought) tolerance.

Drought resistance can assayed according to any of a number of well-known techniques. For example, plants can be grown under conditions in which less than optimum water is provided to the plant. Drought resistance can be determined by any of a number of standard measures including turgor pressure, growth, yield, and the like. In some embodiments, the methods described in the Example section below can be conveniently used.

### EXAMPLES

The following examples are offered to illustrate, but not to limit the claimed invention.

Water stress caused by drought and soil salinity is an important environmental factor that limits the productivity and distribution of plants. The cellular and molecular mechanisms of plant responses to water stress have been analyzed extensively (Shinozaki, K. et al., Curr. Opin. Plant Biol. 6:410-41 (2003); Xiong, L. et al., Plant Physiol. 133:29-3 (2003); Zhu, J. K. Annu. Rev. Plant Biol. 53:247-273 (2002)). Water stress can induce the accumulation of the phytohormone abscisic acid (ABA) (Xiong, L. et al., Plant Physiol. 133:29-3 (2003)). ABA plays a vital role in triggering stomatal closure, which reduces transpirational water loss and constitutes an essential part of plant drought tolerance (Shinozaki, K. et al., Curr. Opin. Plant Biol. 6:410-41 (2003); Xiong, L. et al., J. Biol. Chem. 277:8588-859 (2002); Zhu, J. K. Annu. Rev. Plant Biol. 53:247-273 (2002)). Analysis of Arabidopsis thaliana mutants has defined several ABA response loci that encode proteins such as protein phosphatases and kinases, which greatly affect guard cell movement (Finkelstein, R. R. et al., Plant Cell 14:(Suppl.):S15-S45 (2002); Schroeder, J. I. et al., Nature 410:327-33 (2001) Xiong, L. et al., J. Biol. Chem. 277:8588-859 (2002)).

Recent studies indicate that transcripts of protein-coding genes are regulated at all steps of RNA metabolism, from transcription initiation to RNA processing (Sims, R. J., III et al., Genes Dev. 18:2437-246 (2004)). A great deal of information about plant transcriptional regulators that bind the promoters to initiate gene transcription in response to water stress has been collected (Zhang, J. Z. et al., Plant Physiol. 135:615-621 (2004)). In contrast, much less is known about proteins involved in RNA processing (Kuhn, J. M. et al., Curr. Opin. Plant Biol. 6:463-46 (2003)). Nevertheless, recent studies point to a central role of RNA processing in regulating ABA sensitivity and osmotic stress responses. The RNA-binding protein FCA was reported to be an ABA receptor, although it appears to function in ABA regulation of flowering rather than in seed dormancy or drought tolerance (Razem, F. A. et al., Nature 439:290-29 (2006)). ABH1, a cap-binding protein, functions in early ABA signaling (Hugouvieux, V. et al., Cell 106:477-48 (2001)). A recessive mutation in the SAD1 gene encoding an Sm-like snRNP required for mRNA splicing, export, and degradation rendered plants hypersensitive to ABA and drought (Xiong, L. et al., Dev. Cell 1:771-78 (2001)). The Arabidopsis HYL1 gene encodes a nuclear double-stranded RNA-binding protein. A knockout mutation of the HYL1 gene caused abnormal development, increased sensitivity to abscisic acid, and reduced sensitivity to auxin and cytokinin (Lu, C. et al., Plant Cell 12:2351-236 (2000)). HYL1 controls gene expression likely through microRNA-mediated gene regulation, although the targeted genes related to ABA sensitivity are still unknown (Han, M. H. et al., Proc. Natl. Acad. Sci. USA 101:1093-1098 (2004)). AKIP1 isolated from Vicia faba is a single-stranded RNA-binding protein which can bind to a dehydrin mRNA after phosphorylation by an ABA-activated protein kinase (Li, J. et al., Nature 418:793-79 (2002)). The CRYOPHYTE/LOS4 gene encoding a DEAD (SEQ ID NO:7) box RNA helicase is essential for mRNA export, and the cryophyte mutant is hypersensitive to ABA during seed germination (Gong, Z. et al., Plant Cell 17:256-267 (2005); Gong, Z. et al., Proc. Natl. Acad. Sci. USA 99:11507-11512 (2002)). The double-stranded RNA-binding protein FRY2/CPL1 negatively regulates ABA and osmotic stress responses possibly through modulating RNA polymerase II activity by dephosphorylating Ser-5 of its C-terminal domain (Koiwa, H. et al., Proc. Natl. Acad. Sci. USA 99:10893-1089 (2002); Koiwa, H. et al., Proc. Natl. Acad. Sci. USA 101:14539-1454 (2004); Xiong, L. et al., Proc. Natl. Acad. Sci. USA 99:10899-1090 (2002)). Transcriptional elongation mediated by RNA polymerase II is a pivotal process in gene regulation, is highly regulated in eukaryotes by numerous factors in mRNA biogenesis and maturation, and is an emerging topic of active study in biology (Sims, R. J., III et al., Genes Dev. 18:2437-246 (2004)).

We report here the isolation and characterization of the Arabidopsis ABA-overly sensitive 1 (abo1) mutant and map-based cloning of ABO1. The abo1 mutant was isolated on the basis of its drought-resistant phenotype. The abo1 mutation enhances ABA sensitivity in both seedling growth and stomatal closure. ABO1 is a new allele of ELO2 (Nelissen, H. et al., Proc. Natl. Acad. Sci. USA 102:7754-775 (2005)), which encodes a homolog of yeast Iki3/Elp1/Tot1 or human IκB kinase-associated protein (IKAP), collectively the largest subunit of Elongator, a complex with roles in secretion, tRNA modification, and mRNA transcription elongation (Cohen, L. et al., Nature 395:292-296 (1998); Frohloff, F. et al., EMBO J. 20:1993-2003 (2001); Gilbert, C. et al., Mol. Cell. 14:457-464 (2004); Huang, B. et al., RNA 11:424-436 (2005); Kim, J. H. et al., Proc. Natl. Acad. Sci. USA 99:1241-124 (2002); Otero, G. et al., Mol. Cell. 3:109-11 (1999); Rahl, P. B. et al., Mol. Cell. 17:841-85 (2005); Slaugenhaupt, S. A. et al., Curr. Opin. Genet. Dev. 12:307-31 (2002); Yajima, H. et al., Biosci. Biotechnol. Biochem. 61:704-70 (1997)). These findings suggest that Elongator is important for plants to respond to ABA and drought exposure and that ABO1/ELO2 may play a vital role in ABA signal transduction pathways.

### Experimental Procedures

### Plant Growth Conditions

Plants were grown in 340-ml pots filled with a mixture of peat/forest soil and vermiculite (3:1) in a greenhouse at 22° C., with light intensity of 50 μmol m-2 s-1 and 70% rH under long-day conditions (16-h-light/8-h-dark cycle). Seedlings were germinated and grown on Murashige and Skoog (MS) medium (M5519; Sigma) supplemented with 3% (wt/vol) sucrose and 0.8% agar under the same growth conditions.

Isolation of the abo1 Mutant, Growth Conditions, and Genetic Analysis.

The isolation of the abo1-1 mutant of Arabidopsis thaliana (Columbia gl1 background) was performed by use of a water loss screening system. To identify mutants, ethyl methyl sulfonate (EMS)-mutagenized M2 seeds were sown on MS medium. Four-day-old seedlings were transferred to soil and grown for 2 weeks with sufficient watering, and then water was withheld. The abo1-1 mutant was identified as a plant surviving the drought treatment, while other plants around it wilted and died. The abo1-1 mutant was backcrossed to the wild-type plant in the original background, and the resulting F1 seedlings as well as F2 progeny from self-fertilized F1 plants were evaluated in a drought stress assay.

Three T-DNA insertion mutants (Columbia background, SALK database accession no. SALK_{—}004690, SALK_{—}011529, and SALK_{—}084199) were obtained from the Arabidopsis Biological Resource Center (Columbus, Ohio). Allelic tests done by crossing the four mutants (the abo1 mutant and the three T-DNA insertion mutants) revealed that they were allelic and so were named abo1-1 (EMS-mutagenized mutant) and abo1-2, abo1-3, and abo1-4 (T-DNA insertion mutants). All subsequent physiological and phenotypic analyses were carried out using the abo1-1 mutant that had been backcrossed to the wild type four times to remove other mutations.

### Genetic Mapping.

abo1-1 mutant plants (Columbia gl1 background) were crossed to Landsberg wild-type plants. A total of 1,077 abo1-1 mutant plants were selected from the self-fertilized F2 population by the leaf water loss assay combined with the identification of the abnormal stomatal development phenotype. DNA was isolated from individual mutant plants and analyzed for recombination events with simple sequence length polymorphism (SSLP) markers. The following primer pairs for SSLP markers that are polymorphic between Columbia gl1 and Landsberg were developed: for F8L15, forward primer 5′-CGGTAATACCTATGGAGCCGCCG-3′ (SEQ ID NO:8) and reverse primer 5′-GCGCATGGTACCGCTAATGGCAG-3′ (SEQ ID NO:9); for MUA22, forward primer 5′-GGAGAGACTGATGGACGCCATTTG-3′ (SEQ ID NO:10) and reverse primer 5′-GTCCTCATCAAGGGGCTGCAGAGG-3′ (SEQ ID NO:11); for T24H18, forward primer 5′-CAGTGCATGGTTTGCATGGGAA-3′ (SEQ ID NO:12) and reverse primer 5′-TTTACGCAGGACATGTTTCCTCTC-3′ (SEQ ID NO:13); for T6114, forward primer 5′-AAGGACCAGCGTGGCTCAAG-3′ (SEQ ID NO:14) and reverse primer 5′-AATCACTCACTGCCTCTTTGGAGG-3′ (SEQ ID NO:15); and for MXE10, forward primer 5′-CGTCAGGGTGCTGCTTTTCTC-3′ (SEQ ID NO:16) and reverse primer 5′-GTGCCTGCACATTGATCACCATC-3′ (SEQ ID NO: 17).

### Stomatal Aperture Bioassays.

Stomatal closing assays were conducted as described previously (Pei, Z. M. et al., Plant Cell 9:409-42 (1997)). Rosette leaves were floated in solutions containing 50 μM CaCl2, 10 mM KCl, 10 mM MES [2-(N-morpholino)ethanesulfonic acid]-Tris, pH 6.15, and exposed to light (150 μmol m-2 s-1) for 2 h. Subsequently, ABA was added to the solution at 0.5 to 10 μM to assay for stomatal closing. After ABA treatment for 2 h, stomatal apertures were measured as described previously (Pei, Z. M. et al., Plant Cell 9:409-42 (1997)). Values are means ±standard errors (SE) (n=30). Significance (P<0.05) was assessed by Student's t test.

### Stomatal Development Assays.

Epidermal strips from rosette leaves of 3-week-old seedlings of wild-type and mutant plants were examined for stomata under a light microscope (B5-223 IEP; Motic China Group Co., Ltd.). For scanning electron microscopy, rosette leaves of 3-week-old seedlings of wild-type and mutant plants were fixed as described previously (Burton, R. A. et al., Plant Cell 12:691-706 (2000)), and stomata were observed under a scanning electron microscope (S-570; Hitachi, Japan).

### Water Loss Measurements and Determination of ABA Content.

Rosette leaves of mutant and wild-type plants growing under normal conditions for 3 weeks were detached and weighed immediately on a piece of weighting paper and then placed on a laboratory bench (40% rH) and weighed at designated time intervals. Three replicates were done for each line. The percentage of loss of fresh weight was calculated on the basis of the initial weight of the plants. ABA contents were determined as described previously (Chen, Z. et al., Plant J. 43:273-283 (2005)).

pABO1-GUS Chimeric Construct and Histochemical Analysis.

A promoter fragment of 2,173 bp of the ABO1 gene, defined as pABO1, which contains the promoter region of ABO1, its partial coding region, and the partial coding region of ABO1's upstream gene (MSH12.16), was amplified from Columbia gl1 genomic DNA by PCR with the primer pair 5′-CACCGGAAGGAAGAGAGCTGAAGGGC-3′ (SEQ ID NO:18) (to add CACC at the 5′ end) and 5′-GAGGGTCAGAGGGATTCAGAAGG-3′ (SEQ ID NO: 19). The amplified fragment was cloned into a Gateway Technology system for cloning and expression (Invitrogen), resulting in a transcriptional fusion of the ABO1 promoter and its partial coding region with the GUS coding region. The ABO1 promoter-GUS fusion construct was introduced into Agrobacterium tumefaciens and transferred into plants. Thirty T2 transgenic lines were subjected to β-glucuronidase (GUS) assays. GUS staining was performed as described previously (Shi, H. et al., Plant Cell 14:465-47 (2002)).

### RNA Gel Blot Analysis.

Seedlings grown on MS medium for 3 weeks were transferred to a solution containing 100 μM ABA or no ABA (for control) for 3 h or 5 h. Total RNA was isolated and analyzed as previously described (Chen, Z. et al., Plant J. 43:273-283 (2005)). An RD29A fragment (967 bp) was amplified by PCR with forward primer 5′-GACGAGTCAGGAGCTGAGCTG-3′ (SEQ ID NO:20) and reverse primer 5′-CGATGCTGCCTTCTCGGTAGAG-3′ (SEQ ID NO:21). A fragment (552 bp) of the RD29B gene was amplified by using forward primer 5′-CCGACGGGAACTCATGATCAGTTC-3′ (SEQ ID NO:22) and reverse primer 5′-CACTTCCACCTCCTTTGTAGCCG-3′ (SEQ ID NO:23). A COR47 fragment (413 bp) was amplified by PCR with forward primer 5′-GAAGCTCCCAGGACACCACGAC-3′ (SEQ ID NO:24) and reverse primer 5′-CAGCGAATGTCCCACTCCCAC-3′ (SEQ ID NO:25). An ABI1 fragment (517 bp) was amplified by PCR with forward primer 5′-CGCAGGTCCTTTCAGGCCATTC-3′ (SEQ ID NO:26) and reverse primer 5′-GCCATGGCCGTCGTAAACAC-3′(SEQ ID NO:27). These gene fragments were used as probes. A tubulin gene was used as a loading control.

Elongator Cross-Complementation Studies with Yeast and Plant.

For phenotypic complementation assays, the yeast tot1/elp1[Delta] mutant LFY3, deleted for the Elongator subunit 1 gene (TOT1/ELP1) (Jablonowski, D. et al., Mol. Biol. Cell 15:1459-146 (2004)), was transformed with pDJ98, a vector for galactose-regulated expression of the plant Elongator subunit 1, ABO1/ELO2 (Nelissen, H. et al., Proc. Natl. Acad. Sci. USA 102:7754-775 (2005)). pDJ98 construction involved PaeI and SacI restriction of pMD18.T, Klenow fill-in of the resulting 4-kb ABO1/ELO2 cDNA, and subcloning into SmaI-restricted pBluescript (Stratagene) to yield pDJ79. Following pDJ79 restriction by SalI, the ABO1/ELO2 cDNA was cloned into SalI-cut pYEX-GAL, a pYEX-BX (Clontech) expression vector derivative with its CUP1 promoter replaced by the GAL1 promoter. The latter process involved BamHI/HindIII replacement of the GAL1 promoter fragment from plasmid pRB1438 (kindly provided by Mike Stark, University of Dundee, United Kingdom). Collectively, 2 μm multicopy vector pDJ98 allows galactose-driven expression of ABO1/ELO2, its maintenance is selectable by URA3, and its copy number is amplifiable by use of leu2d, a transcriptionally compromised marker (Spalding, A. et al., J. Gen. Microbiol. 135:1037-104 (1989)).

Sensitivity tests towards endogenous expression of the lethal γ-toxin subunit of zymocin involved LFY3 cells transformed with pDJ98, empty 2 μm vector pYEX-GAL, and pFF14, a 2 μm vector carrying the yeast TOT1/ELP1 gene (Frohloff, F. et al., EMBO J. 20:1993-2003 (2001)). Following subsequent transformations with pHMS14 (a GAL1-γ-toxin expression vector) or pHMS22 (an empty GAL1-promoter control) (Frohloff, F. et al., EMBO J. 20:1993-2003 (2001)), 10-fold serial dilutions of these yeast tester strains were grown on 2% (vol/vol) glucose synthetic complete medium (Sherman, F. Methods Enzymol. 194:3-2 (1991)) lacking tryptophan, leucine, and histidine. The response towards γ-toxin expression was monitored after the strains were shifted onto 2% (vol/vol) galactose medium. Growth proceeded for 3 to 4 days at 30° C. Testing the effects of chemical stress towards growth performance of the LFY3 transformants involved addition of 5 to 10 mM caffeine (Sigma) to complete yeast extract-peptone-dextrose (YPD) media (Sherman, F. Methods Enzymol. 194:3-2 (1991)) and cultivation at 30° C. Thermosensitivity was tested between 30° C. and 39° C.

### Results

Identification of the abo1 mutant.

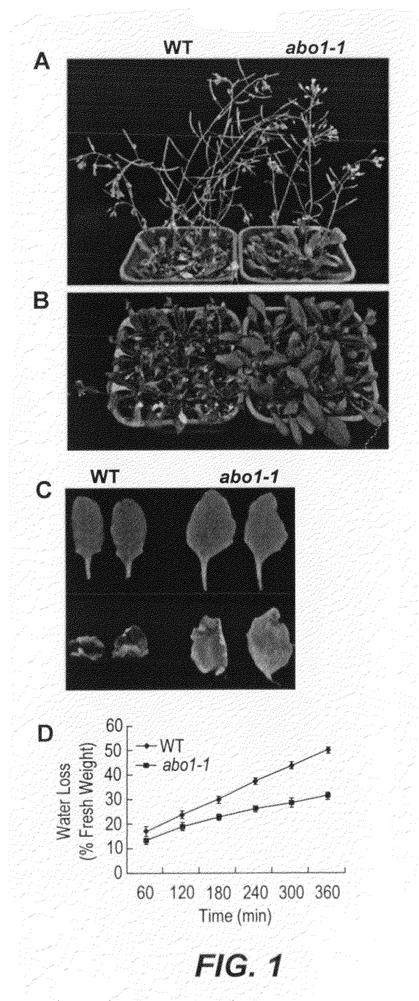

Water was withheld from an EMS-mutagenized population of Arabidopsis (Columbia gl1 background) seedlings grown in a growth room for 2 weeks to screen for mutants with increased drought sensitivity or resistance. In order to recover drought-sensitive mutants, we collected 10 EMS-mutagenized M1 plants as a small pool. Approximately 108 M2 seedlings from each pool were planted in 12 pots filled with soil. We screened approximately 57,000 plants from 534 pools. Putative mutants with drought response phenotypes were scored by the degree of leaf wilting compared with that of neighboring plants. The abo1-1 mutant was identified by its less severe leaf wilting and enhanced drought resistance. FIG. 1A shows wild-type and abo1-1 plants grown for 3 weeks and then treated with drought by withholding water for 11 days. abo1-1 leaves were still turgid, whereas wild-type leaves showed serious wilting. After an additional 5 days, we rewatered the treated plants and found that 100% of abo1-1 plants survived, while all wild-type plants died. We also treated 2-week-old plants in soil by withholding water and obtained a similar result (FIG. 1B), indicating that the abo1-1 plant is more drought resistant in two different growth stages. The drought-resistant phenotype of the abo1 mutant was further evaluated by measuring water loss from detached leaves. As shown in FIGS. 1C and D, detached leaves of the abo1-1 mutant lost water more slowly than did those of the wild-type plants.

In order to determine whether the abo1-1 mutant phenotype is caused by mutation in a single nuclear gene, we crossed the abo1-1 plant with the wild-type plant, and all resulting F1 plants showed the wild-type phenotype under drought stress. An F2 progeny from self-fertilized F1 segregated in an ˜3:1 ratio (198 to 67) between wild-type and abo1-1 mutant phenotypes. These results indicate that abo1-1 has a recessive mutation in a single nuclear gene.

Stomatal Closure and Seedling Growth of the abo1-1 Mutant are Hypersensitive to ABA.

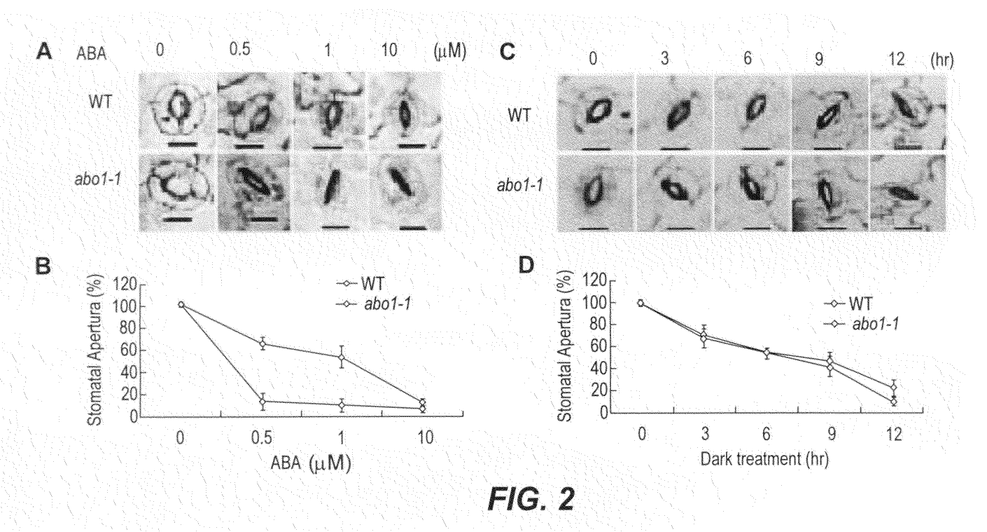

In order to determine the cause of the drought-resistant phenotype of the abo1-1 mutant, we performed experiments to determine whether this phenotype is related to increased sensitivity of stomatal closure to ABA. abo1-1 and wild-type plants were exposed to high humidity for 12 h to open stomata fully. Epidermal peels from these plants were used to analyze stomatal responses to ABA. In response to ABA treatment at different concentrations, the closure of preopened abo1-1 stomata was greatly enhanced compared to that of the wild type (FIGS. 2A and B). Exposure to 0.5 μM ABA enhanced stomatal closure to the same degree in abo1-1 plants as 10 μM ABA did in the wild type (FIG. 2B). Stomata in abo1-1 plants were almost completely closed with 1 μM ABA treatment for 2 h, but the stomata did not completely close even with 20 μM ABA in wild-type plants for the same treatment time (data not shown). By contrast, darkness-induced stomatal closure in abo1-1 plants was similar to that of the wild type (FIGS. 2C and D). These results indicate that the abo1-1 mutation specifically enhances the sensitivity of stomatal closure to ABA.

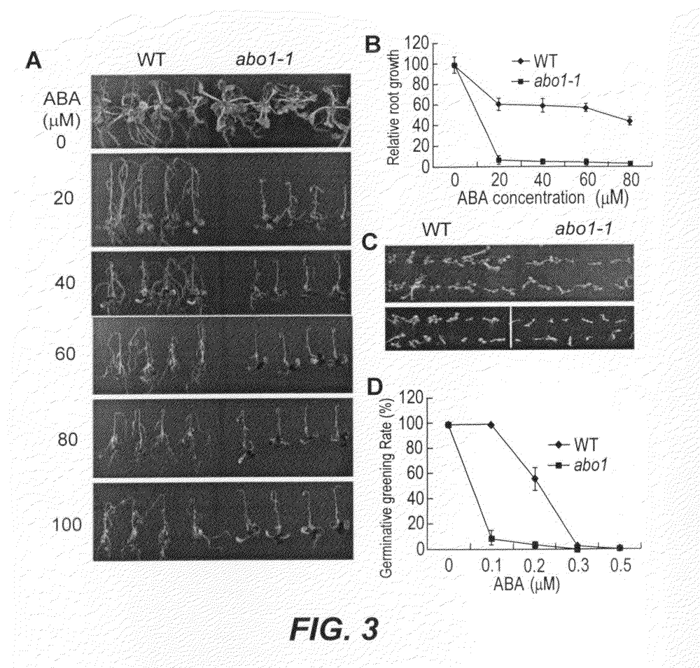

We also analyzed the ABA sensitivities of abo1-plants in seed germination and seedling growth. Seedlings grown for 5 days on MS medium were transferred to MS media containing different concentrations of ABA. One week later, the plants were scored for visible changes. Without ABA, the root growth of abo1-1 plants was slower than that of wild-type plants but they had similar levels of shoot growth. In the presence of different concentrations of ABA, both root and shoot growth of abo1-1 plants were more inhibited than that of the wild type (FIGS. 3A and B). At higher concentrations of ABA (more than 70 μM), abo1-1 leaves showed more anthocyanin pigments, and some cotyledons became chlorotic, which was rarely seen in wild-type plants even at 100 μM ABA. However, supplementation of different concentrations of ABA on agar plates did not produce a difference in abo1 seed germination compared to that of the wild type (FIG. 3C). The postgermination growth of abo1-1 seedlings was more impaired than that of the wild type, as indicated by the ratio of seedlings with green cotyledons to the total number of seedlings after germination for 1 week (FIG. 3D). In order to determine whether abo1 mutants are specifically sensitive to ABA, we also tested the effects of other phytohormones, including methyl jasmonate, salicylic acid, indole-3-acetic acid, gibberellins (GA3), and ethylene, on seedling growth but failed to find any difference between the abo1 mutant and the wild type (data not shown).

The abo1 Mutation Enhances Oxidative Stress Tolerance.

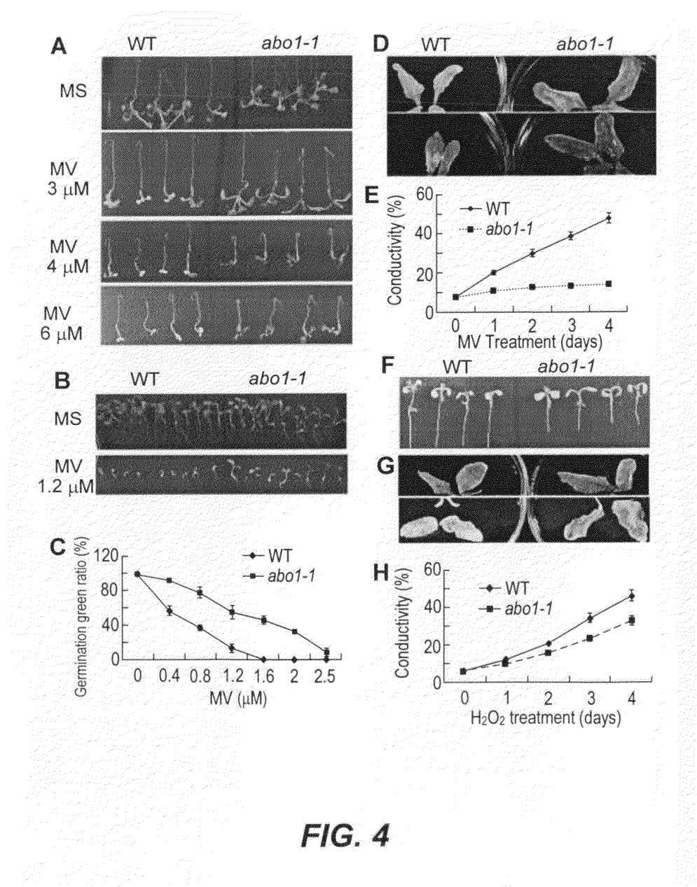

ABA can induce the expression of genes encoding antioxidant enzymes (Guan, L. et al., Plant Physiol. 117:217-224 (1998); Zhu, D. et al., Plant Physiol. 106:173-178 (1994)). ABA also induces the production of reactive oxygen species that serve as a second messenger in ABA signaling in guard cells (Jiang, M. et al., Plant Cell Physiol. 42:1265-127 (2001); Pei, Z. M. et al., Nature 406:731-73 (2000)). In order to test whether the ABA sensitivity of abo1-1 seedlings might be connected with reactive oxygen species, we analyzed the response of abo1-1 mutant plants to oxidative stress. Methyl viologen (MV) (paraquat) inhibits electron transport in the reduction of NADP to NADPH during photosynthesis and thus enhances H2O2 production in chloroplasts under light (Suntres, Z. E. Toxicology 180:65-7 (2002)). Seedlings grown on MS medium for 5 days were transferred to new MS medium supplemented with different concentrations of MV. The abo1-1 seedlings grew well on MS medium supplemented with 3 μM MV, while more than 50% of wild-type seedlings were killed at this concentration (FIG. 4A) after a 5-day treatment. Exposure to 4 μM MV completely killed all wild-type seedlings, but all abo1 mutant seedlings still survived even at 6 μM MV. Seed germination was inhibited to similar extents for both abo1-1 and wild-type plants by various concentrations of MV. After radicles emerged, the young seedlings were more sensitive to MV. FIG. 4B shows that exposure to 1.2 μM MV for 10 days prevented the cotyledon greening of more than 90% of the wild-type seedlings, whereas only 40% of the abo1-1 cotyledons were affected. Dose-response analysis further indicates that abo1-1 mutant seedlings are less sensitive to MV than wild-type seedlings (FIG. 4C).

The leaves taken from plants grown in soil were treated with 1 μM MV. After 5 days, the abo1-1 leaves showed enhanced anthocyanin pigmentation but without any apparent chlorotic symptom. In contrast, wild-type leaves were severely damaged and displayed chlorotic spots or bleaching (FIG. 4D). Electrolyte leakage (conductivity) is often used as an indicator of tissue damage. The abo1-1 leaves treated with 1 μM MV showed substantially less ion leakage than the wild-type leaves (FIG. 4E). Rose Bengal is another reagent that generates H2O2 when exposed to the light. Exposure to 10 μM Rose Bengal for 3 days severely damaged the cotyledons of wild-type seedlings, compared with no chlorotic symptom observed for abo1 mutant cotyledons (FIG. 4F). We also found that wild-type leaves were more damaged than abo1-1 leaves by treatment with 10 mM H2O2 for 5 days (FIG. 4G). This was supported by ion leakage measurements at different time points (FIG. 4H). These results show that abo1-1 mutant plants are more resistant to oxidative stress and suggest a possible connection between the ABA and oxidative stress response phenotypes.

### Regulation of Gene Expression by ABO1.

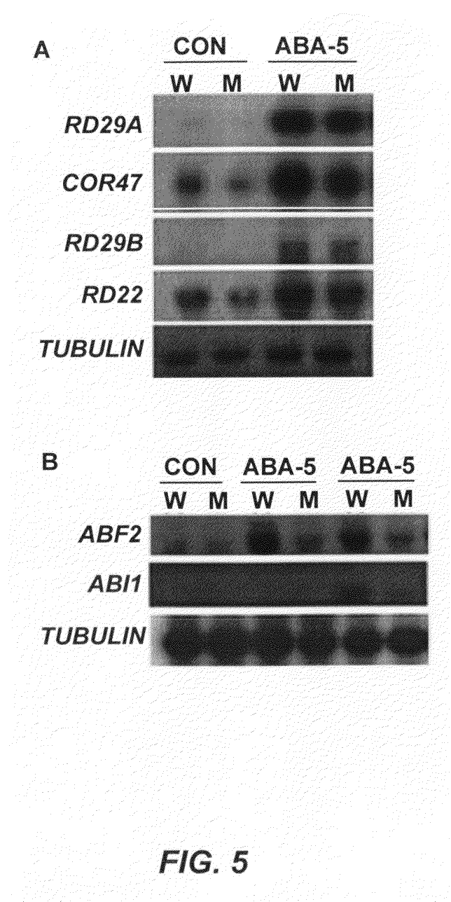

To ascertain whether the ABA response defects of the abo1 mutation are related to an altered expression of ABA- and stress-responsive genes, we conducted Northern blot analysis of different stress-inducible genes. We used the abo1-2 mutant for Northern blot analysis because it is more likely a null allele (see below). The expression of RD29A and COR47 genes is partly ABA independent, while RD22 and RD29B are dependent on ABA (Xiong, L. et al., J. Biol. Chem. 277:8588-859 (2002)). Under our conditions, both COR47 and RD22 are expressed at a low level in wild-type and mutant untreated control samples, but the expression levels of both genes are lower in the mutant than in the wild type (FIG. 5A). In response to 20 μM ABA treatment for 5 h, the expression of all four genes is induced to higher levels in the wild type than in the mutant, although the expression difference is rather small for RD29A and RD29B (FIG. 5A).

The transcription factor ABF2/AREB1 (Kim, S. et al., Plant J. 40:75-87 (2004)) positively regulates the expression of ABA-responsive genes, whereas the protein phosphatase 2C ABI1 negatively regulates ABA responses (Gosti, F. et al., Plant Cell 11:1897-1910 (1999); Merlot, S. et al., Plant J. 25:295-30 (2001)). We found that the expression of ABF2/AREB1 is more induced in the wild type than in the abo1-2 mutant upon ABA treatment (FIG. 5B). The induction of the ABI1 transcript by ABA is lower in the abo1 mutant than in the wild type at 5 h of treatment (FIG. 5B).

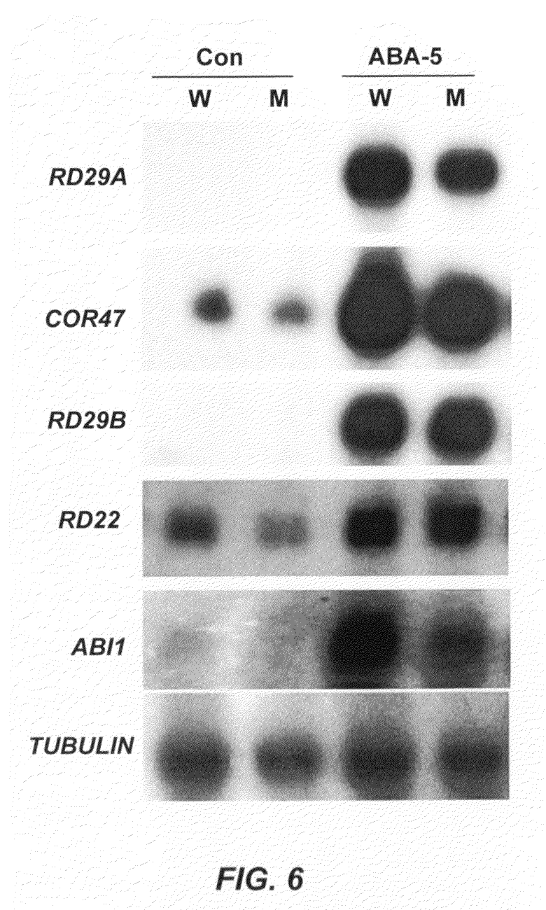

We further compared the expression levels of some of these genes in response to a higher ABA concentration (100 μM). The transcripts of RD29A, COR47, and ABI1 are induced to lower levels in the abo1 mutant than in the wild type, whereas the expression levels of RD29B and RD22 are similar between the abo1 mutant and the wild type (FIG. 6). These results suggest that ABA-responsive genes are less sensitive to ABA induction in the abo1 mutant but that high concentrations of ABA may compensate for the difference for some of the genes.

### ABO1 Appears to Differentially Regulate the Development and Growth of Two Adjacent Pairs of Guard Cells.

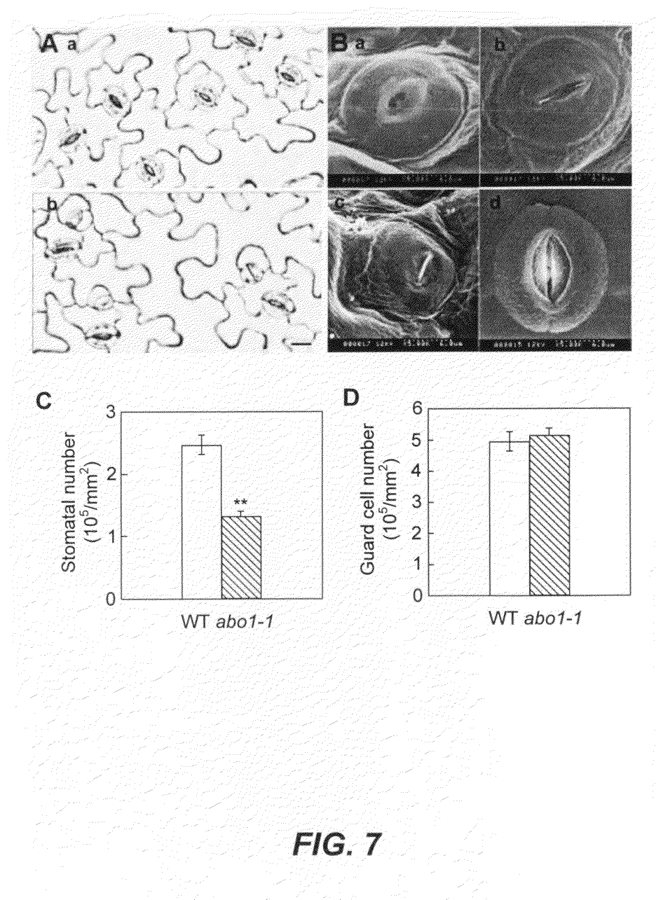

Drought resistance could be contributed by both ABA sensitivity of stomatal movement and stoma number. A stoma is formed through one or more asymmetric cell divisions followed by the symmetric division of the guard mother cell (Nadeau, J. A. et al., Trends Plant Sci. 8:294-29 (2003)). During our experiments on stomatal movement, we noticed that the number of stomata with pores that could be observed under a light microscope in the abo1-1 mutant was only about half of that in the wild type (FIGS. 7A and C). Some pairs of guard cells did not form normal stomata or formed stomata with very small pores (FIG. 7B). However, the total number of guard cells that form stomata with or without pores in the abo1-1 plant is almost the same as that in the wild-type plant (FIG. 7D). The abo1 mutation appears to affect only the development and growth of guard cells and their adjacent pavement cells and not the division and differentiation of their precursor cells. These results suggest that, in addition to increased ABA sensitivity of fully developed stomata, the drought-resistant phenotype of the abo1-1 mutant may be caused in part by the reduced number of stomata on the leaf surface.

### Map-Based Cloning of ABO1.

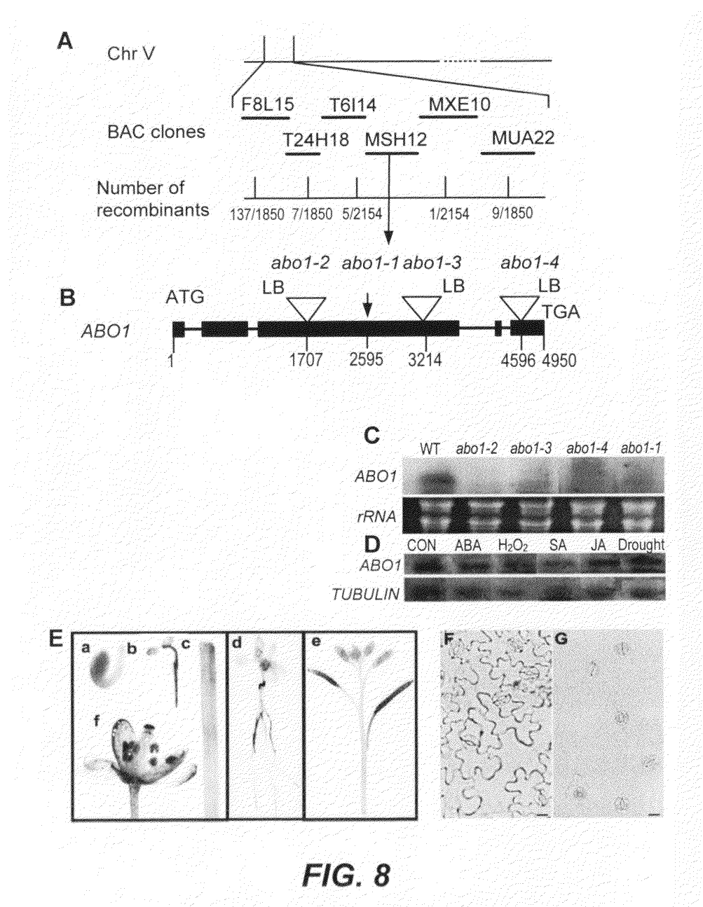

We used a map-based cloning strategy to identify the mutation responsible for the abo1 mutant phenotype. The abo1-1 mutant in the Columbia background was crossed with wild-type plants in the Landsberg background. The phenotypes of the resultant F2 seedlings were determined, and DNA was extracted from each plant and examined with SSLP markers designed according to information from Cereon Genomics (http://www.Arabidopsis.org). Initial mapping located abo1 to the top of chromosome V. Further mapping positioned ABO1 to within six bacterial artificial chromosome clones: F8L15, T24H18, T6I14, MSH12, MXE10, and MUA22. Continued mapping delimited ABO1 to a region within the bacterial artificial chromosome clones T6I14, MSH12, and MXE10 (FIG. 8A). Several predicted open reading frames were amplified by PCR and sequenced. A single nucleotide mutation from G2595 (counting from the first putative ATG of the genomic sequence) to A was found in abo1-1 in the third predicted exon of the putative gene At5g13680 (MSH12.15). The mutation was predicted to change amino acid Trp746 (encoded by TGG) to a stop codon (encoded by TAG) and to result in a C-terminal truncation mutant lacking almost half of the polypeptide sequence of the ABO1 protein (FIG. 8B).

We searched the SALK collection database and obtained three independent insertion mutants (SALK_{—}004690, SALK_{—}011529, and SALK_{—}084199) from the Arabidopsis Stock Center (http://www.Arabidopsis.org). Sequencing analysis confirmed the insertion sites provided by the Salk Institute Genome Analysis Laboratory. T-DNA insertions of SALK_{—}004690, SALK_{—}011529, and SALK_{—}084199 were detected before nucleotides 1707, 3214, and 4596 (counting from the first putative ATG of the genomic sequence), respectively. We renamed SALK_{—}004690, SALK_{—}011529, and SALK_{—}084199, respectively, abo1-2, abo1-3, and abo1-4 (FIG. 8B). Northern blot analysis using a probe amplified from nucleotides 1916 to 3037 of ABO1 (counting from the first putative ATG of the genomic sequence) revealed that the expression of the ABO1 gene was detected in the wild type but not in the mutant alleles abo1-1, abo1-2, abo1-3, and abo1-4 (FIG. 8C). We crossed abo1-1 with abo1-2, abo1-3, and abo1-4, and all of the resulting F1 plants showed the abo1-1 drought resistance phenotype. All three T-DNA insertion mutants have, per leaf area, about half the number of guard cells with pores that can be seen under a light microscope compared to the wild type. We also checked the ABA sensitivities of abo1-2, abo1-3, and abo1-4 and found that all of the mutants showed a phenotype similar to that of abo1-1. Taken together, these data demonstrate that ABO1 is the gene mutated in the different abo1 alleles.

### ABO1/ELO2 Encodes a Homolog of Yeast Elongator Subunit Elp1/Iki3/Tot1.

ABO1 is anew allele of ELO2, which is predicted to encode a protein of 1,319 amino acids with significant similarity to the largest subunit of the yeast Elongator complex, Elp1/Iki3/Tot1 (Frohloff, F. et al., EMBO J. 20:1993-2003 (2001); Nelissen, H. et al., Proc. Natl. Acad. Sci. USA 102:7754-775 (2005)). ABO1 shares 50% identity and 68% similarity in the entire amino acid sequence with a putative rice IKI3 (GenBank accession no. NP_{—}910712), 26% identity and 45% similarity in the entire amino acid sequence with the human homolog of yeast Iki3, IKAP (GenBank accession no. AAG43369), and 27% identity and 44% similarity in the entire amino acid sequence with the fission yeast Iki3 (GenBank accession no. NP_{—}595335).

In order to analyze the ABO1/ELO2 gene expression pattern, transgenic Arabidopsis plants expressing an ABO1/ELO2 promoter-ABO1/ELO2 partial coding region-GUS reporter gene were analyzed for GUS activity. The GUS gene was expressed in roots, hypocotyls, stems, leaves, flowers, and siliques (FIG. 8E, panels b to f) but was not detected at the earlier stage of seed germination, which is consistent with the lack of mutant effect on ABA inhibition of seed germination (FIG. 8E, panel a). Consistent with the role of ABO1/ELO2 in guard cells, GUS activity was detected in guard cells of isolated epidermal peels (FIGS. 8F and G). It appears that GUS staining is greater in one of the two adjacent pairs of stomata which are usually formed in Arabidopsis. However, because of the low GUS expression and development sequence of the guard cells, it is difficult to differentiate the GUS expression levels of later-formed guard cells from those of earlier-formed guard cells. The GUS expression pattern is consistent with our observed mutant phenotypes. Nevertheless, the GUS expression pattern may not fully reflect the expression pattern of the endogenous ABO1 gene since the ABO1 promoter fragment used for the GUS experiment does not include potential regulatory sequences that may be present in the introns or other parts of the gene.

To determine whether ABO1/ELO2 gene expression is induced by stress, we performed Northern blot experiments using total RNA extracted from 2-week-old seedlings treated with different stresses. The ABO1/ELO2 transcript was not induced by drought or H2O2 or by treatment with exogenous hormones such as ABA, salicylic acid, or jasmonic acid (FIG. 8D), which indicates that ABO1/ELO2 is not a stress-inducible gene.

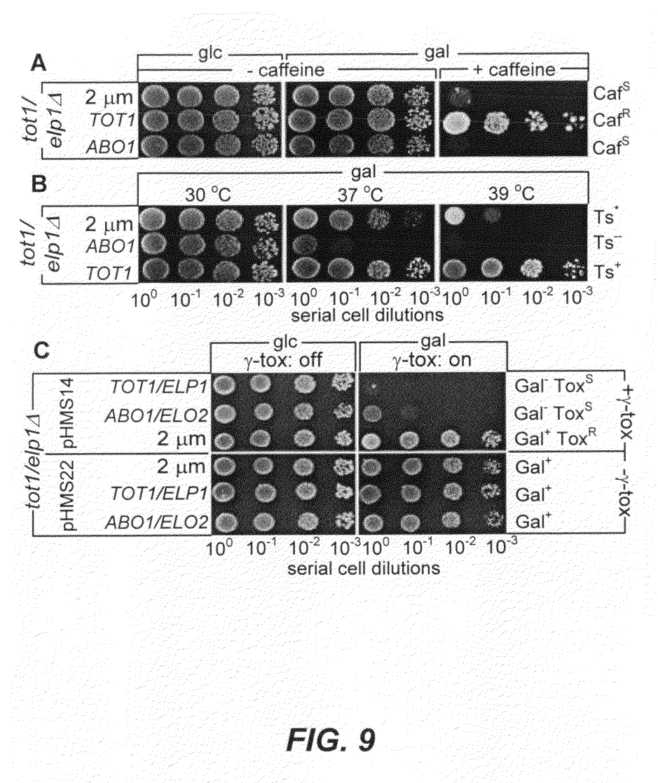

In order to study the plant ABO1/ELO2 gene, we asked whether its expression complements a yeast tot1/elp1 [Delta] mutant lacking the Elongator subunit 1 gene homologous to ABO1/ELO2. In yeast, loss of Elongator elicits sensitivity to thermal and chemical stress and causes resistance to cell death by the zymocin toxin complex (Frohloff, F. et al., EMBO J. 20:1993-2003 (2001); Jablonowski, D. et al., Mol. Microbiol. 42:1095-110 (2001)). Intriguingly, while caffeine sensitivity of the tot1/elp1[Delta] strain remained unaltered at 30° C. under galactose induction of the ABO1/ELO2 gene (FIG. 9A), cell viability of ABO1/ELO2 expressors became progressively compromised at 37° C. and ceased completely at 39° C. (FIG. 9B) in the absence of caffeine. This effect is not linked to thermosensitivity of the Elongator mutant (Frohloff, F. et al., EMBO J. 20:1993-2003 (2001)), as tot1/elp1[Delta] cells carrying empty vector proved to be significantly more thermotolerant than ABO1/ELO2 expressors. As expected, the wild-type yeast gene TOT1/ELP1 efficiently complemented tot 1/elp1[Delta] cells to restore wild-type thermotolerance at 39° C. and caffeine resistance (FIGS. 9A and B). Hence, under conditions known to interfere with Elongator deficits, ABO1/ELO2 expression is not able to improve cell viability. Contrast this with complementation of γ-toxin resistance due to galactose induction of the ABO1/ELO2 gene (FIG. 9C). Based on normal performance in the presence of pHMS22, a vector devoid of the zymocin subunit gene, we consider the growth arrest of ABO1/ELO2 expressors by the γ-toxin (FIG. 9C) to be fully ascribable to ABO1/ELO2 gene expression. Hence, the ability of ABO1/ELO2 to confer γ-toxin sensitivity in tot1/elp1[Delta], an otherwise γ-toxin-resistant Elongator mutant (Frohloff, F. et al., EMBO J. 20:1993-2003 (2001)), strongly indicates that the plant gene ABO1/ELO2 substitutes for this zymocin-relevant function of the yeast Elongator subunit 1. Whether the ABO1/ELO2 protein assembles into a chimeric Elongator complex is not yet known but appears likely since, in yeast, zymocin toxicity requires a structurally integrated six-subunit complex (Fichtner, L. et al., Mol. Microbiol. 45:817-826 (2002); Frohloff, F. et al., J. Biol. Chem. 278:956-961 (2003)).

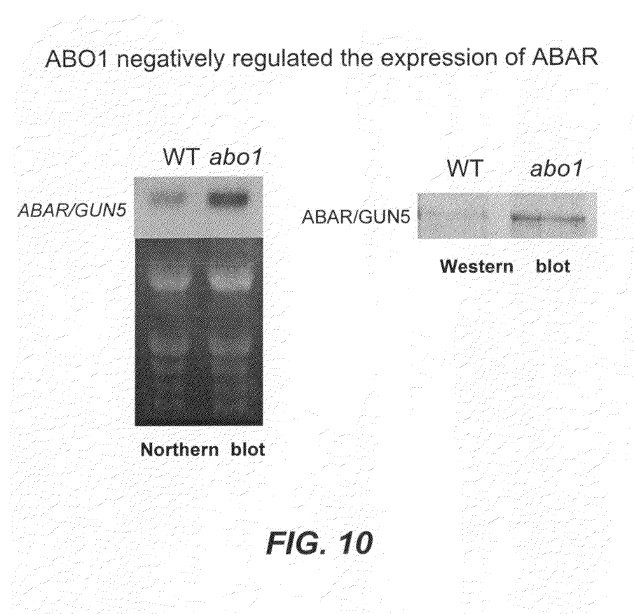

Increased expression of ABAR (also known as GUN5) was observed in abo1 plants compared to wildtype plants. Increased expression was observed at both the mRNA and protein level, as shown in FIG. 10. ABAR has recently been identified as an ABA receptor (see, Shen et al., Nature 443:823-826 (2006). The results here are consistent with ELO2 acting as a negative regulator of ABAR.

### DISCUSSION

Several genetic loci important in ABA signaling have been identified previously based on seed germination sensitivity to ABA (Finkelstein, R. R. et al., Plant Cell 14:(Suppl.):S15-S45 (2002)) and abnormal bioluminescence emitted from plants carrying the RD29A luciferase gene under ABA treatment in Arabidopsis (Chinnusamy, V. et al., Sci. STKE 2002:PL10 (2002)). We have succeeded in isolating mutations through directly screening the drought-resistant or leaf-wilting plants growing on soil under drought stress conditions (Chen, Z. et al., Plant J. 43:273-283 (2005)). The abo1 mutations isolated in this study greatly increased drought tolerance. abo1 mutants show ABA hypersensitivity in the inhibition of seedling growth and the promotion of stomatal closing. Furthermore, the abo1 mutant is more resistant to oxidative stress, which may be related to its ABA hypersensitivity and increased drought tolerance. Interestingly, mutations in ABO1/ELO2 also influence the development of guard cells, resulting in stomata reduced to half the number in the wild type. These results indicate that ABO1/ELO2 represents a unique mechanism for modulating drought tolerance in Arabidopsis.

Studies with yeast and animals have demonstrated the importance of Elongator, a histone acetyltransferase complex, in controlling gene expression at the elongating stage of transcription and potentially mRNA processing (Gilbert, C. et al., Mol. Cell. 14:457-464 (2004); Kim, J. H. et al., Proc. Natl. Acad. Sci. USA 99:1241-124 (2002)). In yeast, holo-Elongator comprises two subcomplexes: core-Elongator, consisting of subunits Elp1-3/Tot1-3, and the smaller Elp4-6/Tot5-7 module (Frohloff, F. et al., EMBO J. 20:1993-2003 (2001); Krogan, N. J. et al., Mol. Cell. Biol. 21:8203-821 (2001); Winkler, G. S. et al., J. Biol. Chem. 276:32743-3274 (2001)). Elp1/Tot1 is the largest subunit with homology to the human IKAP, which can cause a severe neurodegenerative disorder called familial dysautonomia when mutated (Anderson, S. L. et al., Am. J. Hum. Genet. 68:753-758 (2001)). In Arabidopsis, putative homologs of all six yeast Elongator subunits were predicted based on comparative genomics (Nelissen, H. et al., Plant Cell 15:639-65 (2003)). Three Arabidopsis loci homologous to the yeast Elongator gene ELP1, ELP3, and ELP4 subunits and responsible for phenotypes of the elongata2 (elo2), elo3, and elo1 mutants have been cloned recently (Nelissen, H. et al., Proc. Natl. Acad. Sci. USA 102:7754-775 (2005)).

Our findings that induction of ABO1/ELO2 from the GAL1 promoter restored sensitivity of a tot1/elp1[Delta] Elongator mutant towards growth inhibition by the zymocin γ-toxin subunit suggest functional Elongator conservation. In line with previous reports that zymocin-induced cell death requires holo-Elongator (Fichtner, L. et al., Mol. Microbiol. 45:817-826 (2002); Frohloff, F. et al., J. Biol. Chem. 278:956-961 (2003)), complementation by ABO1/ELO2 implies that tot 1/elp1 [Delta] cells expressing the plant gene are likely to assemble an Elongator chimera that accommodates ABO1/ELO2 and that supports Elongator function in yeast. Based on differential phenotypic displays, i.e., complemented zymocin resistance, unaltered caffeine sensitivity, and enhancement of thermosensitivity, cross-complementation by ABO1/ELO2, however, is considered to be partial. Although the capability of ABO1/ELO2 to rescue caffeine sensitivity of tot1/elp1[Delta] cells by upregulating ABO1/ELO2 expression was not investigated, we cannot exclude the possibility that full Elongator competence may require excess ABO1/ELO2 levels in yeast. In support, complementation of the γ-toxin phenotype required plasmid-coupled ABO1/ELO2 expression under the control of leu2d that amplifies plasmid copy number (Spalding, A. et al., J. Gen. Microbiol. 135:1037-104 (1989)). Alternatively, differential phenotypes may indicate that some Elongator functions are conserved from yeast to plants while others are not. Our observation that ABO1/ELO2 induction enhanced thermosensitivity points to proliferation-relevant aspects that may distinguish plant from yeast Elongator. Congruently, the mechanism by which Elongator affects cell proliferation reportedly differs between yeast and plants (Nelissen, H. et al., Proc. Natl. Acad. Sci. USA 102:7754-775 (2005)) and hELP3, human Elongator subunit 3, hardly replaced the yeast homolog (Li, F. et al., Mol. Genet. Genomics 273:264-27 (2005)). Whether Elongator's roles in transcription (Gilbert, C. et al., Mol. Cell. 14:457-464 (2004); Otero, G. et al., Mol. Cell. 3:109-11 (1999)), secretion (Rahl, P. B. et al., Mol. Cell. 17:841-85 (2005)), or tRNA modification (Huang, B. et al., RNA 11:424-436 (2005)) are hijacked by zymocin is under study. Recent reports that zymocin particularly targets Elongator-dependent tRNA species support the latter option (Jablonowski, D. et al., Mol. Microbiol. 59:677-68 (2006); Lu, J. et al., RNA 11:1648-165 (2005)) and provide a strong case to study whether Elongator's novel role in tRNA modification is conserved among yeast, plants, and mammals.

Recent studies identified several Arabidopsis mutations that affect ABA signaling by controlling RNA metabolism (Kuhn, J. M. et al., Curr. Opin. Plant Biol. 6:463-46 (2003)). Among them, an ABH1 mutation encodes an mRNA cap-binding protein which, together with CBP20, forms a heterodimeric nuclear cap-binding complex (Hugouvieux, V. et al., Cell 106:477-48 (2001); Papp, I. et al., Plant Mol. Biol. 55:679-68 (2004)), and a SAD1 mutation encodes a multifunctional Sm-like protein which is a component of snRNPs functioning in pre-RNA splicing and mRNA transport and degradation (Xiong, L. et al., Dev. Cell 1:771-78 (2001)). Mutations in either ABH1 or SAD1 or CBP20 render plants hypersensitive to ABA in seed germination and seedling growth (Hugouvieux, V. et al., Cell 106:477-48 (2001); Papp, I. et al., Plant Mol. Biol. 55:679-68 (2004); Xiong, L. et al., Dev. Cell 1:771-78 (2001)). Interestingly, mutations in ABO1/ELO2 lead to similar ABA sensitivity of seedling growth but cause no clear change in response to other plant hormones. All of these phenotypes are also observed with ABH1 and SAD1 mutants. Because all three genes (SAD1, ABO1/ELO2, and ABH1) are involved in different stages during mRNA processing, together these studies suggest that the RNA-processing machinery or part of it is intimately involved in early ABA signaling for stress tolerance (Hugouvieux, V. et al., Cell 106:477-48 (2001); Kuhn, J. M. et al., Curr. Opin. Plant Biol. 6:463-46 (2003); Xiong, L. et al., Dev. Cell 1:771-78 (2001)).

Our Northern blotting results indicated that mutations in ABO1/ELO2 affect the expression of some stress-inducible genes. Although abo1 mutants are hypersensitive to ABA in both stomatal closing and seedling growth, the expression levels of ABA-responsive genes did not show hypersensitivity to exogenous ABA in the abo1 mutant. In fact, upon 20 μM ABA treatment the expression levels of stress-responsive marker genes RD29A, RD29B, RD22, and COR47 as well as ABF2/AREB1 and ABI1 are lower in the abo1 mutant. ABO1/ELO2 is a single-copy nonessential gene in Arabidopsis. As discussed above, one of Elongator's functions is to facilitate mRNA transcription elongation by RNA polymerase II (Gilbert, C. et al., Mol. Cell. 14:457-464 (2004); Kim, J. H. et al., Proc. Natl. Acad. Sci. USA 99:1241-124 (2002); Otero, G. et al., Mol Cell 3:109-11 (1999)). The mutations in ABO1/ELO2 would decrease the transcript levels of its target mRNAs. It has also been shown that both ABH1 and SAD1 have minor effects on global gene expression and that only a limited number of genes are altered by the abh1 or sad1 mutations (Hugouvieux, V. et al., Cell 106:477-48 (2001); Xiong, L. et al., Dev. Cell 1:771-78 (2001)). At present, the molecular mechanism underlying the differential gene expression effects of the mRNA processing-related mutations is not known. However, based on the recent report that ABA can affect RNA processing by binding to the RNA-binding protein FCA (Razem, F. A. et al., Nature 439:290-29 (2006)), it is possible that ABA may directly modulate these RNA processing steps by binding to one or more of the RNA processing factors.

Another interesting phenotype of the abo1 mutant is a reduced stoma number. In abo1 mutants, the total number of stomata observed on the leaf surface was half of the number in the wild type, but the total numbers of guard cells were similar. The abundance of stomata is an important factor influencing water use efficiency. Several genes affecting guard cell patterning and development have been isolated recently (Berger, D. et al., Genes Dev. 14:1119-1131 (2000); Bergmann, D. C. et al., Science 304:1494-1497 (2004); Boudolf, V. et al., Plant Cell 16:945-955 (2004); Nadeau, J. A. et al., Trends Plant Sci. 8:294-29 (2003); Shpak, E. D. et al., Science 309:290-29 (2005); Von Groll, U. et al., Plant Cell 14:1527-153 (2002)). Mutations in SDD, YODA, and TMM genes showed increased stoma number in mutant plants, which indicates that these genes are negative regulators in guard cell formation. Results suggest that SDD, YODA, and TMM function upstream and FLP, CDKB1, and FAMA function downstream in the guard cell development signaling pathway (Bergmann, D. C. et al., Science 304:1494-1497 (2004); Boudolf, V. et al., Plant Cell 16:945-955 (2004)). The guard cell phenotypes caused by ABO1/ELO2 mutations are different from those of the guard cell-related mutants in the literature. ABO1/ELO2 affects only the growth and development but not the division and differentiation of the pairs of guard cells originating from satellite meristemoid mother cells. Another important difference is that the abo1 mutations impair not only stomatal development but also stomatal sensitivity to ABA, whereas other stomatal developmental mutants do not have defects in ABA sensitivity (Berger, D. et al., Genes Dev. 14:1119-1131 (2000); Bergmann, D. C. et al., Science 304:1494-1497 (2004); Boudolf, V. et al., Plant Cell 16:945-955 (2004); Nadeau, J. A. et al., Trends Plant Sci. 8:294-29 (2003); Shpak, E. D. et al., Science 309:290-29 (2005); Von Groll, U. et al., Plant Cell 14:1527-153 (2002)). Because ABO1/ELO2 is suggested to influence the mRNA elongation process, ABO1/ELO2 may not directly participate in controlling the growth and development of guard cells. Instead, ABO1/ELO2 may regulate another gene(s) that plays a role in guard cell growth and development. The fact that abo1 mutants are hypersensitive to ABA-induced stomatal closure suggests that some genes responsible for stomatal closure could also be involved in regulating guard cell growth. Our results suggest that the growth and development of pairs of guard cells originating from meristemoid mother cells and satellite meristemoid mother cells are modulated by different genes, possibly through different signaling pathways.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of enhancing abscisic acid sensitivity in a plant, the method comprising introducing an recombinant expression cassette into a plant, wherein the expression cassette comprises a promoter operably linked to a polynucleotide comprising at least 20 contiguous nucleotides complementary or identical to a contiguous sequence in a cDNA encoding an endogenous polypeptide at least 50% identical to SEQ ID NO:2, SEQ ID NO:4 or SEQ ID NO:6 in the plant, wherein the promoter is heterologous to the polynucleotide, thereby reducing expression of the polypeptide in the plant, wherein the plant has increased abscisic acid sensitivity compared to an otherwise identical plant lacking the expression cassette.

2. The method of claim 1, wherein the polynucleotide comprises at least 50 contiguous nucleotides complementary or identical to a contiguous sequence in the cDNA.

3. The method of claim 1, wherein the polynucleotide comprises at least 200 contiguous nucleotides complementary or identical to a contiguous sequence in the cDNA.

4. The method of claim 1, wherein the polypeptide is at least 95% identical to a protein selected from the group consisting of SEQ ID NO:2, SEQ ID NO:4 and SEQ ID NO:6.

5. The method of claim 1, wherein the plant has improved drought tolerance compared to an otherwise identical plant lacking the expression cassette.

6. The method of claim 1, wherein the polynucleotide comprises at least 20 contiguous nucleotides complementary or identical to a contiguous sequence in SEQ ID NO:1, SEQ ID NO:3, or SEQ ID NO:5.

7. The method of claim 1, wherein the promoter directs expression to guard cells of the plant.

8. The method of claim 1, wherein the promoter is constitutive.

9. The method of claim 1, wherein the promoter is inducible.

10. The method of claim 1, wherein the promoter is tissue-specific.

11. A recombinant expression cassette comprising a promoter operably linked to a polynucleotide comprising at least contiguous 20 nucleotides complementary or identical to a contiguous sequence in a cDNA encoding an endogenous polypeptide at least 50% identical to SEQ ID NO:2, SEQ ID NO:4 or SEQ ID NO:6 in the plant, wherein the promoter is heterologous to the polynucleotide, and wherein introduction of the expression cassette into a plant results in enhanced abscisic acid sensitivity in the plant compared to an otherwise identical plant lacking the expression cassette.

12. The recombinant expression cassette of claim 11, wherein introduction of the expression cassette into a plant results in improved drought tolerance in the plant compared to an otherwise identical plant lacking the expression cassette

13. The recombinant expression cassette of claim 11, wherein the polynucleotide comprises at least 50 contiguous nucleotides complementary or identical to a contiguous sequence in the cDNA.

14. The recombinant expression cassette of claim 11, wherein the polynucleotide comprises at least 200 contiguous nucleotides complementary or identical to a contiguous sequence in the cDNA.

15. The recombinant expression cassette of claim 11, wherein the polypeptide is at least 95% identical to a protein selected from the group consisting of SEQ ID NO:2, SEQ ID NO:4 and SEQ ID NO:6.

16. The recombinant expression cassette of claim 11, wherein the polynucleotide comprise at least 20 contiguous nucleotides complementary or identical to a contiguous sequence in SEQ ID NO:1, SEQ ID NO:3, or SEQ ID NO:5.

17. The recombinant expression cassette of claim 11, wherein the promoter directs expression to guard cells of the plant.

18. The recombinant expression cassette of claim 11, wherein the promoter is constitutive.

19. The recombinant expression cassette of claim 11, wherein the promoter is inducible.

20. The recombinant expression cassette of claim 11, wherein the promoter is tissue-specific.

21. A transgenic plant comprising a recombinant expression cassette, the recombinant expression cassette comprising a promoter operably linked to a polynucleotide comprising at least 20 contiguous nucleotides complementary or identical to a contiguous sequence in a cDNA encoding an endogenous polypeptide at least 50% identical to SEQ ID NO:2, SEQ ID NO:4 or SEQ ID NO:6 in the plant, wherein the promoter is heterologous to the polynucleotide, and wherein the plant has enhanced abscisic acid sensitivity compared to an otherwise identical plant lacking the expression cassette.

22. The plant of claim 21, wherein the plant has improved drought tolerance compared to an otherwise identical plant lacking the expression cassette.

23. The plant of claim 21, wherein the polynucleotide comprises at least 50 contiguous nucleotides complementary or identical to a contiguous sequence in the cDNA.

24. The plant of claim 21, wherein the polynucleotide comprises at least 200 contiguous nucleotides complementary or identical to a contiguous sequence in the cDNA.

25. The plant of claim 21, wherein the polypeptide is at least 95% identical to a protein selected from the group consisting of SEQ ID NO:2, SEQ ID NO:4 and SEQ ID NO:6.

26. The plant of claim 21, wherein the plant has improved drought tolerance compared to an otherwise identical plant lacking the expression cassette.

27. The plant of claim 21, wherein the polynucleotide comprise at least 20 contiguous nucleotides complementary or identical to a contiguous sequence in SEQ ID NO:1, SEQ ID NO:3, or SEQ ID NO:5.

28. The plant of claim 21, wherein the promoter is constitutive.

29. The plant of claim 21, wherein the promoter is inducible.

30. The plant of claim 21, wherein the promoter is tissue-specific.

31. The plant of claim 21, wherein the promoter directs expression in guard cells.

32. A seed, flower, leaf or fruit from the plant of claim 21.


SEQUENCE LISTING (27 sequences)

SEQ ID NO: 1 - DNA, 4121 bp, Arabidopsis thaliana
  thale cress abscisic acid (ABA)-overly sensitive 1 (ABA-OVERLY SENSITIVE 1, ABO1), elongata 2 (ELONGATA 2, ELO2), ABO1/ELO2, Elongator subunit, transcriptional elongation factor, MSH12.15, CHR5v07142002, At5g13680, IKI3 family
atgaaaaatttgaagcttttctcggaggttcctcagaacatccaattgcattcgactgaagaagttgtacagttcgcagcttatgacatcgaccaaagtcgcttatttttcgcatcttctgctaatttcgtttacgcccttcagctctcttccttccaaaatgaaagcgctggggcaaagagtgctatgcctgtggaggtctgcagtattgatattgagcctggggatttcatcacggctttcgattatctcgcggagaaggagtctctattaattgggacttctcatgggcttttgctagtgcataatgtggagagtgatgtgactgagctagttggaaatatagaaggaggtgtcaagtgtatctcgcctaatccgactggagatctacttggattgattactggtcttgggcagttgcttgtaatgacttatgattgggctttgatgtacgagaaggcgcttggagaggttcctgaaggtggttacgtacgtgaaacgaatgatttgtctgtaaattgtgggggtatttccatttcctggaggggtgatggaaagtattttgcgactatgggtgaggtttatgagtctggttgtatgtctaagaagattaagatatgggaaagtgattccggtgctttgcagtcttcttcagaaacaaaagaatttacgcaggggattcttgaatggatgccgagtggtgctaaaatcgcagctgtttataagaggaagtcagatgatagcagtccatcgattgctttctttgagaggaatggacttgagagaagctcgtttagaattggtgaaccagaagatgctacggaatcatgtgaaaatctgaaatggaattctgcatcagatcttcttgcaggggttgttagttgtaaaacatatgatgccattagggtgtggttctttagcaacaaccactggtacctgaaacaggagattaggtatccaagggaagctggggtaacagtcatgtgggatccaacaaagccactgcagttaatatgttggactctgtcagggcaggtgtctgttcgccactttatgtgggtaacagcggtcatggaggactcaacagcctttgtcatagataactccaagatactcgtgacaccactatctttgtccttgatgccacctcctatgtatttgttcagccttagtttttcttctgctgtcagagatatagcatattactcaaggaattctaagaattgtctggctgtgtttttgtcagatggaaacctgtcttttgttgagttccctgcacctaatacttgggaagatctcgaaggcaaagacttcagtgtggaaatttcagactgtaaaacagcactgggctcttttgtacatcttctatggttggatgtccactcacttctgtgtgtctccgcttatggttctagccacaacaagtgtctctcttcgggagggtatgacacagagcttcatggttcttatttacaagaagttgaagtagtctgtcatgaggatcatgttcctgatcaagtgacttgctctggctttaaggctagcattactttccagacacttctcgaatcaccggtcctggctcttgcctggaatcctagtaagcgtgattcagcctttgtggagtttgagggtgggaaagtgcttggatatgcgtcaaggtcagaaattatggaaactcgtagtagtgatgatagtgtatgttttccatcgacttgtccttgggtgagggtggcacaggttgatgctagtggggtgcataaacccttgatatgtgggcttgatgatatgggtagactgtccatcaacgggaagaacctatgtaataactgtagcagtttctcattttattcagagttggccaatgaagtagttacccatctgattattttgaccaaacaggattttctctttattgtcgataccaaggatgtactgaatggagatgtggcacttggaaatgtgttctttgttattgatggtagaaggcgagacgaggaaaatatgagctatgttaatatttgggaaagaggtgcaaaagtgataggagtcctcaatggagatgaagctgctgtgatattacaaactatgcgggggaatctcgaatgcatatatcccagaaagctggtcctctcttccattacgaatgcgttagctcagcaacggttcaaagatgcatttaacttggtaaggcgtcatagaatcgactttaatgttatcgtggatctatatgggtggcaggcatttctacagtcagccgtagcatttgttgagcaagtaaacaatttgaaccatgttacagaatttgtttgtgccatgaagaatgaagatgttacagaaacgctgtacaaaaagttttccttttccaaaaagggagacgaggtttttcgtgtgaaagatagttgcagtaacaaagtatcatcagttctacaggcaattaggaaggcccttgaagaacatatcccagaaagtccatcaagggaactctgtattttgaccactcttgcccgaagtgatccaccagctatcgaagaatctcttttgaggataaaatctgttcgtgaaatggagttgctaaactcttctgatgatataaggaaaaagtcttgtccatctgcagaagaagccttgaaacatcttctgtggttattggattctgaggctgtctttgaagctgccttaggtctttatgatctgaaccttgcagctattgtggcactcaattcccagcgtgatccaaaagaatttttaccttatctccaggagctggaaaaaatgcccgaatccctgatgcatttcaagattgacattaaattgcagcgttttgatagtgctcttaggaacattgtatcagcaggggttggctactttcctgattgcatgaacttaataaagaaaaaccctcaacttttccctctgggcctcctgctaattactgatcctgagaagaaactagttgttctagaggcttgggcagatcacctcattgatgaaaaacgttttgaggatgctgctactacatacctatgttgctgtaaactggaaaaggcttcaaaggcatatcgtgaatgtggtgactggagtggggtactccgagtgggggcgctaatgaaattaggtaaagatgagatcttgaaattggcatatgaactctgtgaagaggtcaatgctcttgggaaaccagcggaagcagcaaaaatagcgcttgagtattgcagcgatataagcggtggaattagtttgcttatcaatgcgagggaatgggaagaggctttaagggttgcatttttgcatacagcagatgatagaatctcagtggtgaaaagttctgctttggagtgtgctagtggtctagtaagtgaattcaaagaatcaatagaaaaggtaggaaaatatttgactcgctatctagctgtccggcagagacgattattgcttgcagcaaagctcaaatccgaggaacgatctgttgttgatcttgacgatgacacagcttcagaagcaagtagcaacctgagtggaatgagcgcctatactttggggacaaggagaggttctgctgcttcagtcagctcaagcaacgctactagcagggcaagagatttgaggcgccagagaaagagtgggaaaatccgtgctggcagtgcgggtgaggagatggctcttgttgatcatttgaaaggtatgcgtatgaccgatggaggaaagcgagagctgaagtcgttgttgatatgtctggtaacacttggtgaaatggagtctgcacagaagttgcaacagactgcagagaatttccaggtctcccaagtagcagctgtagagctagcacacgatacagtgtccagcgagagcgtagatgaagaagtttattgctttgaacgttacgctcagaagacgagatccacagcacgagattcagatgcctttagctggatgcttaaggtcttcataagcccatgaaaatctctacatctgtttttgactgaataagcttgtgctttttaaacatcaaaaccttcctctaccgttttgtatctttcctattgttactgtactatgagattaaggtgcatctgctacatttttgttcattgctaatgcttacattccgattcaaaagg

SEQ ID NO: 2 - protein, 1319 aa, Arabidopsis thaliana
  thale cress abscisic acid (ABA)-overly sensitive 1 (ABA-OVERLY SENSITIVE 1, ABO1), elongata 2 (ELONGATA 2, ELO2), ABO1/ELO2, Elongator subunit, transcriptional elongation factor, MSH12.15, CHR5v07142002, At5g13680, IKI3 family
MKNLKLFSEVPQNIQLHSTEEVVQFAAYDIDQSRLFFASSANFVYALQLSSFQNESAGAKSAMPVEVCSIDIEPGDFITAFDYLAEKESLLIGTSHGLLLVHNVESDVTELVGNIEGGVKCISPNPTGDLLGLITGLGQLLVMTYDWALMYEKALGEVPEGGYVRETNDLSVNCGGISISWRGDGKYFATMGEVYESGCMSKKIKIWESDSGALQSSSETKEFTQGILEWMPSGAKIAAVYKRKSDDSSPSIAFFERNGLERSSFRIGEPEDATESCENLKWNSASDLLAGVVSCKTYDAIRVWFFSNNHWYLKQEIRYPREAGVTVMWDPTKPLQLICWTLSGQVSVRHFMWVTAVMEDSTAFVIDNSKILVTPLSLSLMPPPMYLFSLSFSSAVRDIAYYSRNSKNCLAVFLSDGNLSFVEFPAPNTWEDLEGKDFSVEISDCKTALGSFVHLLWLDVHSLLCVSAYGSSHNKCLSSGGYDTELHGSYLQEVEVVCHEDHVPDQVTCSGFKASITFQTLLESPVLALAWNPSKRDSAFVEFEGGKVLGYASRSEIMETRSSDDSVCFPSTCPWVRVAQVDASGVHKPLICGLDDMGRLSINGKNLCNNCSSFSFYSELANEVVTHLIILTKQDFLFIVDTKDVLNGDVALGNVFFVIDGRRRDEENMSYVNIWERGAKVIGVLNGDEAAVILQTMRGNLECIYPRKLVLSSITNALAQQRFKDAFNLVRRHRIDFNVIVDLYGWQAFLQSAVAFVEQVNNLNHVTEFVCAMKNEDVTETLYKKFSFSKKGDEVFRVKDSCSNKVSSVLQAIRKALEEHIPESPSRELCILTTLARSDPPAIEESLLRIKSVREMELLNSSDDIRKKSCPSAEEALKHLLWLLDSEAVFEAALGLYDLNLAAIVALNSQRDPKEFLPYLQELEKMPESLMHFKIDIKLQRFDSALRNIVSAGVGYFPDCMNLIKKNPQLFPLGLLLITDPEKKLVVLEAWADHLIDEKRFEDAATTYLCCCKLEKASKAYRECGDWSGVLRVGALMKLGKDEILKLAYELCEEVNALGKPAEAAKIALEYCSDISGGISLLINAREWEEALRVAFLHTADDRISVVKSSALECASGLVSEFKESIEKVGKYLTRYLAVRQRRLLLAAKLKSEERSVVDLDDDTASEASSNLSGMSAYTLGTRRGSAASVSSSNATSRARDLRRQRKSGKIRAGSAGEEMALVDHLKGMRMTDGGKRELKSLLICLVTLGEMESAQKLQQTAENFQVSQVAAVELAHDTVSSESVDEEVYCFERYAQKTRSTARDSDAFSWMLKVFISP

SEQ ID NO: 3 - DNA, 4590 bp, Oryza sativa
  rice (japonica cultivar-group, Nipponbare (GA3) cultivar) Os07g0563700 gene, IKI3 family
acccgccgccgcggcccatcaactccggcgagctccacccgccgccctccaccctcctccctccctccgtctccgtctctccccggatctcctcccgcggccggaagggatccaccggccaccggcggcggggggatgaagaacctaaaggtggttacccggatagcgcagaagcttcagctccagctcgacggggagaccgtggtagtctccgccgtcgacgccgagcgccgccgcgccttcttcgtctcctccgagaacttcctctactccgtcgacctccccgcccccacccagcaatcactgcaatggagcgaaagtaccctagactctgatgcagaggaggttgttcttgaacctggagattacatagttgccatggattatctaatggagaaggagtctttgctgcttggttcatcaaccggttgtctattattgtataatgtggatgagaaaactactgaagttgtgggacgactggagggtggtgtgaagaccattgcttctagtcctgatggggcccttatttctgttacgaccgggtttggacaattgctcatcatgacaaacgattgggaagtgctgtttgagacttctctcgaccctcattgtgatcttacaggtgatatcaacagtcccaatggtcacatccaaagttctatttcttggcggggtgatggaaagttttttgctactcttggtgggcttgaagggagctctcagaaacttactatttgggaacgtgaatcaggaaatatacattcttcttcagataccaaggcttttataggagcatcacttgattggatgccaagtggagccaaggttgccactgcccatgaccggaagacagagggaaagtgccctctcattgtattctatgaaaagaatggcttagaaaggagccacttttccattgatgagccagcagaggctgtcatccaagctttaaaatggaactgcaattctgaactcttagctgctctagtctcttgtggccagtatgatgttatcaaagtatggtcctgcagtaacaatcactggtacttgaaacaagaactgcgatacacaaagaaagaaggggtgaggttctattgggatcaaacaaaaccaatgcatctcatctgctggacattaggtggtcaggttattacgcacaggtttgcttggactactgctgtcagtgagacttcaattgcgcttgttattgatggttcccatgtccttgtaactcctctcagtttgggtctcatgccaccccccatgtctttgttccatcttacatttccttgtgctgtgaatgaggtttcttttctatcctataactcgaaaaaccatattgctgcttatctttctaatggcagcttgtgtgtgtcggtacttccggtggcagatacttgggaagagtttgaaggcagtggcataagtgttgacccttgcttttctgagtcaacattgaataactatatgcacctgacttggattgatacgtgtaccttgatcggcatttgttgccgcgctgatgccttctcttcaacaccaatgaggtccagtgaagctagtagccttctagagaaaaatgactcgccatattttatcaatgagattgaacttgtatgttccgaggattctttgctaggttcagcctgttcatcaggttggcaggcaaaaatatcgaagaaaatgcctctgcaggctgcagttattggaatatctcaaaatccagcaaaagaaggttcagcctttattcagttaagtggaggaagggttgttgaatactgctcaaaagtgaatttgtttagaatgtctgccccggtacaagttagtgaaacaggttccgaccatacttttccaacatcatgcccttggatgactgctgttcaatgccatgaaaatggaatggttaggacacttctctttggacttgatgatagcagcaagttgcatgtgggcaaaaggttattaagcagtaactgtagcagcttcacattctattccagtgcttatggagcaaaagagcaggttgtgacccacttgcttgtgactactaagcaggatcttttattcatcgtggatatcagtgagattttacttaaaaatggcgaagtgacaaccgatagccatatcaggagtcatcctcgaggaaagcaaagcaaagagcatatcactgtatgggaaaagggggcaaagctgattggtgttctccatggtgatgaagcagctgtcataatgcaaaccacccgtggtaacttggagtgtatctaccccaggaagctggtccttgtttcaattgttcaggcactggttcagggacgttttagagatgcatttgacatggtaaggcgccatcggattgatttcaatatggttgttgactactgtggctggaaatcttttatgaagtcagcggcagattttgttaaacaagtcaataaccttagccacatcactgaatttgtttgttcaataaagaatgagaatgtcagcagcaaattgtatgagacctacatatcatttcctgatcactgtgcaacctctgtggctgatactgtaaatactcatggtctgttgtcagacaacaaagtcacatctgtactgatggcaattcgaaaagcccttgaggtgcaggtggaagaaagctcatcaagagaactctgcatactgactactttagcccgtagcgaacctcctctgttggaggaagcactgaatagaataaaagttatccgagaactggaacttcttggggttgatgatgccaggagaaagctctacccatctgctgaagagtccctgaagcacttgctatggttaaccgaaccagaagctgtttttaatgctgctttgggactgtatgatctgaatctttctgctatagtagctttaaattcccaaaaagatccaaaagagttccttccttttctcaagagtcttgaatgccttcctcctgctattatgaagtacacagttgatttaagactcggaagatatgaaagtgctctcaagaacattgtttctgctggcgacgagtatcatgaggactgtatgaaactcctcaacgctaaccctcaattgttccctttggggctccagttattcactgacccagataaaaggcatcaaattcttgaggcatggggtgatcaactttctgaagagaaatgctttgcagatgctgcaataacttatcaatgctgttcgtcgtatcagaaatctttgaaggcgtatcgtgattgtggggattggagaggtgtgttcactgttgcaggtcttctgaagtttaaaaaggaagaaattcttcaactagcacatgacctatgtgatgagttccaagcacttgggaaacctggagatgctgctaaaatagcactggagtattgctcagatgtcgatagaggtgttggttactttatcacggcaagagaatgggaagaagctcttcgggtagcttatatgcacagcaggcaagatctggttgatactgttagagatgcagctttggaatgttctgctttgctgatttctgagtatcaggaaggattgctaaaggtgggcaaatatctggcccgttatgttgctgtacggcagaggagactgtcacttgcagccaagctccagtctgaagaccgatttatggatgttgaagacgacagcatttcagaagtcagcactagcttcagcgaaatgagtgcatataccacaaggtcaacaaaggaatctagtgcttccgtcatatctagcagtgccagcaagtcaagaggggcaaggcgacaaaagaaaggtggcaagatacgagctggaagccctggggaggagatggcacttgtggagcatctcaaagggatgtctctgacctccggtgctctaactgagcttaaaagcctacttgtggttctaatacaactgggaagagaagaaaccgcccgtcaggtgcagctggctggagacaattttgaggtatctcaaagggctgcagtcaagttggctgaggatactgtatccaacgacaagatagacgagaatgcacatacgcttgagaattacgtgaaaatgctgagagctcatcagtcagctgatagtgaaaccacctgttggcgaatcaaagcattgtcgccgccttggaccggggtatactccaattcacatgagcatgctgaatgttgatgagtggtaagcagcggtaactgttggaatggaattgaacgagaggcctcaacaatgattgccatatcattctgtggcggctgtcaagggtaacccggctatatatcgatcgcagccaattcaggatatttatggtgttctgccacttgggtggattgttacgtcaaagtggtgaaacatttttgcttataaatcaggatgttggaggccgcagccgagcccgcttgagctttttgctgcttccatttgtggtataattttgatcgagtagttatatgcatggaggaataaaaagttacatcatctgttcatctgaaaattgggtgttgtagacttgtagtgaatagtgattgtgagacatttaactatttgccacttttaagatgtggcatttaacagttggaagaagtcgtgttgttttcc

SEQ ID NO: 4 - protein, 1343 aa, Oryza sativa
  rice (japonica cultivar-group, Nipponbare (GA3) cultivar) Os07g0563700 gene, IKI3 family
MKNLKVVTRIAQKLQLQLDGETVVVSAVDAERRRAFFVSSENFLYSVDLPAPTQQSLQWSESTLDSDAEEVVLEPGDYIVAMDYLMEKESLLLGSSTGCLLLYNVDEKTTEVVGRLEGGVKTIASSPDGALISVTTGFGQLLIMTNDWEVLFETSLDPHCDLTGDINSPNGHIQSSISWRGDGKFFATLGGLEGSSQKLTIWERESGNIHSSSDTKAFIGASLDWMPSGAKVATAHDRKTEGKCPLIVFYEKNGLERSHFSIDEPAEAVIQALKWNCNSELLAALVSCGQYDVIKVWSCSNNHWYLKQELRYTKKEGVRFYWDQTKPMHLICWTLGGQVITHRFAWTTAVSETSIALVIDGSHVLVTPLSLGLMPPPMSLFHLTFPCAVNEVSFLSYNSKNHIAAYLSNGSLCVSVLPVADTWEEFEGSGISVDPCFSESTLNNYMHLTWIDTCTLIGICCRADAFSSTPMRSSEASSLLEKNDSPYFINEIELVCSEDSLLGSACSSGWQAKISKKMPLQAAVIGISQNPAKEGSAFIQLSGGRVVEYCSKVNLFRMSAPVQVSETGSDHTFPTSCPWMTAVQCHENGMVRTLLFGLDDSSKLHVGKRLLSSNCSSFTFYSSAYGAKEQVVTHLLVTTKQDLLFIVDISEILLKNGEVTTDSHIRSHPRGKQSKEHITVWEKGAKLIGVLHGDEAAVIMQTTRGNLECIYPRKLVLVSIVQALVQGRFRDAFDMVRRHRIDFNMVVDYCGWKSFMKSAADFVKQVNNLSHITEFVCSIKNENVSSKLYETYISFPDHCATSVADTVNTHGLLSDNKVTSVLMAIRKALEVQVEESSSRELCILTTLARSEPPLLEEALNRIKVIRELELLGVDDARRKLYPSAEESLKHLLWLTEPEAVFNAALGLYDLNLSAIVALNSQKDPKEFLPFLKSLECLPPAIMKYTVDLRLGRYESALKNIVSAGDEYHEDCMKLLNANPQLFPLGLQLFTDPDKRHQILEAWGDQLSEEKCFADAAITYQCCSSYQKSLKAYRDCGDWRGVFTVAGLLKFKKEEILQLAHDLCDEFQALGKPGDAAKIALEYCSDVDRGVGYFITAREWEEALRVAYMHSRQDLVDTVRDAALECSALLISEYQEGLLKVGKYLARYVAVRQRRLSLAAKLQSEDRFMDVEDDSISEVSTSFSEMSAYTTRSTKESSASVISSSASKSRGARRQKKGGKIRAGSPGEEMALVEHLKGMSLTSGALTELKSLLVVLIQLGREETARQVQLAGDNFEVSQRAAVKLAEDTVSNDKIDENAHTLENYVKMLRAHQSADSETTCWRIKALSPPWTGVYSNSHEHAEC

SEQ ID NO: 5 - DNA, 4602 bp, Vitis vinifera
  European grape contig VV78X064444.4, Pinot noir cultivar, clone ENTAV 115, locus VITISV_010008, transcriptional elongator hypothetical protein, IKI3 family
atgcgctatcaagagagagggttagtgcacaatttaggaataatcgcatgtatgttaragagtaagttgaatcgccttcttctcccatccggtgtcagacggacagaattcgggatgcgagacatccgaagaaggtgggaatgtcggccggataggattccagatgcgagacatccggagaaggtggaatgttggccggatagaattccggatgacaggattccggatgtgagacattcggagaaggtgggatgtcggcggacaggattccggatgcgagacatccrgagaaggtggaatgtcgactkgatagaattcctgatgtgggacatcyggagaagggtgggacatcggccggacagaatttccggatgtragacatccggagaaggtgggatgtygacggacaggattccggatgtgagacatctggagaaggtggtggaatgtcggccggatagaattccggatgtgggacatccgaagaaggtgggaatgtcggccggacagaattccggatgtgagacatccggcgaaggctgggacgccactctttcctttgwtgttcactggtcaacaagcatggcttgcccaaccactctctactgctgcacgcctgctgcagccggcatcaagccctcagcttcctggctgccagtcccatctggccgggattcatggttcttcacactccaaaaggtaacaccacttcccatgaacagttacttctcatcaaaaccagatgggcaaaatggtcctttcaatattcacccgctttattcagatctctctctgaatctggagttgagatcagccgaagaagttgtctctttctctgcgttcgatattgaacgcaaccgcttggtttttgcttcttccgataatttcatatacaccacgcagcttccttcatcacaaaatgaaagggtatggggcaaaacctcttcatattccaaggttgagcctattgatttggaacctggggacttcattactgcctttgattatctcatggagaaagaagcacttatagtgggaacttctagtgggcttcttttgctgcataatgtggatgataatgcgatagaagttgttggtagggtggagggtggtgtcaagtgcatttcgccaagcccagatggagatctacttggtataataactggttttgggcagatagtagtgatgactcatgattgggatgtgttgtatgagaatacgcttgatgatctgcctgaagatgttgacttaagtgaaccaaccttttcttcctgttatatttcttggcgtggtgatggaaaatactttgtgacactgggggaattgcacacttcttcctcacacaagaagcttaaggtttgggaaagagatacaggggcgctgcatgctgcttcagaatcaaaggctttcatgggaacagttttggattggatgcctagtggagctaaaattgcttctgtttatgacaagaaagttgaaaatgagtgtcccttaatagttttctttgagaggaatggattggaaagaagctcttttagcattaatgagctwactgatgcaaaagtagaaattttaaagtggaattgcagttcagatcttcttgcagctgttgttagatctgaaacatttgactctgttaaaatatggttcttcagcaataatcattggtacctgaaacaagaaattagatacttgagggaggatggagttaaattcatgtggcatccgacaaagccactgcaactgatttgttggactcttggtggagaagtcacagtcaacagctttgtctgggttacagctgttatggagaactcaacagcattagtaattgatgagtccaagatactagcaacccctctttccctttctctaatgccaccaccaatgtatttatttaacctaaagttttcaagtactattagggacattgctttttataccaagaattctaagaatcttttggctgcatttttgtcagatggatgtttgtgtgttgcagagcttcctcctcttgatacttgggaagaactggaagggaaagaattaagtgttgatgcctcctcttctgaaacaatatttgggtcatttgtacatctcatatggttggatgcgcatatacttcttggtgtttctcattttgggttcagtcacagcaattacttttcacaaactccatctagcaaggatatgcttcatgggatcatgtcccaggtttgggaacctgctccaggttggcatgcaaagatcaccaatcaaatacctctagatgggcttgtaattggccttgctcctaatcccacaaaaaagtgttcagcatttgttcagtttgatggtggcaaagtctttgaatatataccaaatttaggcatcatgggaggagctccaaaaactgaggatatgagtttgtcatcatcttgcccttggatgagtgtagttcctgtgggtgacagtggatcatcaaggcctttgctttttgggcttgatgacaatggtaggctgcatgttggtgggaagataatatgtaacaactgcagaagtttctcattttactcaaattcggctgatctagcgataacacatttgattcttgcaactaaacaggatttactctttgttattgacattgatgacattttggatggaaaattggaagtaaaatatgaaaacttcatccatgctggtaataaaagacgcgaagaagataatagaaactttataactatatgggaaagaggtgctaaagttattggtgtccttcatggagatgaagctgctgttatattacaaacagctcgaggaaacctagaatgcatctatccaagaaaattggtcctggcctcaataattaatgctttggttcaaagtcgtttcagagatgggctactcatggtgaggcggcataggatagatttcaatgtcattgtcgaccattgtggttggcaagcctttctccagtcagctgcagagtttgtcaggcaggtaaataatctgagctacatcactgagtttgtttgttccatcaagaatgagactattacagagactttatacaaaaattatatatccctactgtgcccgagggaggctaaagatgtccaagctagagatttcaagggtccaaataataataacaaagtttcttcagttctgatgtctataaggaaggctcttgaggaacaagtaccagaaagtcctgcaagggagctttgcattctgactactctagctcgaagtgatcccccagctcttgaagaagcattggagagaataaaacttatccgggaaatggaactattaggttctgatgatccacgtagaaagtcttacccttctgcagaagaggctctgaagcatcttttgtggttgtctgattctgaggctgtctatgaagcttctttaggcctttatgatttgcaccttgcagctattgtggcattgaactctcaacgggacccaaaagaatttcttccctttcttcaagaactggaacgcatgcctgttcacttgatgcgatataatatcgacattagattgcgcaggtatgagagtgctctcaaacacatcgcttctgcaggggatgcttattatgcagattgtttgaacctcatgaaggaaaatccccaactttttccattgggacttcaactgatcactgatcctgccaagaaaaaagaagtgcttgaggcctggggagatcattttagtgatgaaaaatgctttgaagatgctgctactacttatttgtgctgctctggtttggagaaagctttgaaggcatatcgtgcatgtggtaattggggtggtgtaatgacggttgctggtctccttaaattgggaaaagaagagattgtgcaactggcaaatgagctttgtgaagaactccaggcacttggtaaaccaggggaagctgccaaaattgctctggattactgtggagatgttaaaagtgctatcaatttattggtccagtgcgcgagattgggaggaggcactaagggttgcgtttatgcacaggaaggtctggagaaagtaggaaagtacttagcccgctacttagctgttcggcagagaagattactacttgcagcaaagcttcaatcagaagaccggtcgataaatgatcttgatgatgatactgcttcagaagctagcagtagtttcagtggaatgagtgcttacaccacagggacaaggaaaggctctgctgcttccattagttcaagtacggcaagcaagggaagaggaatgaggcgtcaaaggaatagaggaaaaatccgtgctggaagccctggtgaggagatggctctggtagagcatttgaagggcatgtatcttacccctggagcagagcgtgaacttaaatctctgttggtttcccttgtggtacttggcaaggaagaaatggcgaagaagctacaacgaacaggagaggcctttcaactgtctcaaatggcagcagtcaaactagctgaagatacaatgcctaatgacaacatagatgagtatgcatatactttggagaactacatacagaaattaagaaatgaacagcagtcagatgcttttgtttggcgatctaaagtgctgctttcttcataa

SEQ ID NO: 6 - protein, 1533 aa, Vitis vinifera
  European grape contig VV78X064444.4, Pinot noir cultivar, clone ENTAV 115, locus VITISV_010008, transcriptional elongator hypothetical protein, IKI3 family
MRYQERGLVHNLGIIACMLXSKLNRLLLPSGVRRTEFGMRDIRRRWECRPDRIPDARHPEKVECWPDRIPDDRIPDVRHSEKVGCRRTGFRMRDIXRRWNVDXIEFLMWDIXRRVGHRPDRISGCXTSGEGGMXTDRIPDVRHLEKVVECRPDRIPDVGHPKKVGMSAGQNSGCETSGEGWDATLSFXVHWSTSMACPTTLYCCTPAAAGIKPSASWLPVPSGRDSWFFTLQKVTPLPMNSYFSSKPDGQNGPFNIHPLYSDLSLNLELRSAEEVVSFSAFDIERNRLVFASSDNFIYTTQLPSSQNERVWGKTSSYSKVEPIDLEPGDFITAFDYLMEKEALIVGTSSGLLLLHNVDDNAIEVVGRVEGGVKCISPSPDGDLLGIITGFGQIVVMTHDWDVLYENTLDDLPEDVDLSEPTFSSCYISWRGDGKYFVTLGELHTSSSHKKLKVWERDTGALHAASESKAFMGTVLDWMPSGAKIASVYDKKVENECPLIVFFERNGLERSSFSINELTDAKVEILKWNCSSDLLAAVVRSETFDSVKIWFFSNNHWYLKQEIRYLREDGVKFMWHPTKPLQLICWTLGGEVTVNSFVWVTAVMENSTALVIDESKILATPLSLSLMPPPMYLFNLKFSSTIRDIAFYTKNSKNLLAAFLSDGCLCVAELPPLDTWEELEGKELSVDASSSETIFGSFVHLIWLDAHILLGVSHFGFSHSNYFSQTPSSKDMLHGIMSQVWEPAPGWHAKITNQIPLDGLVIGLAPNPTKKCSAFVQFDGGKVFEYIPNLGIMGGAPKTEDMSLSSSCPWMSVVPVGDSGSSRPLLFGLDDNGRLHVGGKIICNNCRSFSFYSNSADLAITHLILATKQDLLFVIDIDDILDGKLEVKYENFIHAGNKRREEDNRNFITIWERGAKVIGVLHGDEAAVILQTARGNLECIYPRKLVLASIINALVQSRFRDGLLMVRRHRIDFNVIVDHCGWQAFLQSAAEFVRQVNNLSYITEFVCSIKNETITETLYKNYISLLCPREAKDVQARDFKGPNNNNKVSSVLMSIRKALEEQVPESPARELCILTTLARSDPPALEEALERIKLIREMELLGSDDPRRKSYPSAEEALKHLLWLSDSEAVYEASLGLYDLHLAAIVALNSQRDPKEFLPFLQELERMPVHLMRYNIDIRLRRYESALKHIASAGDAYYADCLNLMKENPQLFPLGLQLITDPAKKKEVLEAWGDHFSDEKCFEDAATTYLCCSGLEKALKAYRACGNWGGVMTVAGLLKLGKEEIVQLANELCEELQALGKPGEAAKIALDYCGDVKSAINLLVQCARLGGGTKGCVYAQEGLEKVGKYLARYLAVRQRRLLLAAKLQSEDRSINDLDDDTASEASSSFSGMSAYTTGTRKGSAASISSSTASKGRGMRRQRNRGKIRAGSPGEEMALVEHLKGMYLTPGAERELKSLLVSLVVLGKEEMAKKLQRTGEAFQLSQMAAVKLAEDTMPNDNIDEYAYTLENYIQKLRNEQQSDAFVWRSKVLLSS

SEQ ID NO: 7 - protein, 4 aa, Artificial Sequence
  Description of Artificial SequenceDEAD box
DEAD

SEQ ID NO: 8 - DNA, 23 bp, Artificial Sequence
  Description of Artificial Sequenceforward primer for simple sequence length polymorphism (SSLP) marker F8L15
cggtaatacctatggagccgccg

SEQ ID NO: 9 - DNA, 23 bp, Artificial Sequence
  Description of Artificial Sequencereverse primer for simple sequence length polymorphism (SSLP) marker F8L15
gcgcatggtaccgctaatggcag

SEQ ID NO: 10 - DNA, 24 bp, Artificial Sequence
  Description of Artificial Sequenceforward primer for simple sequence length polymorphism (SSLP) marker MUA22
ggagagactgatggacgccatttg

SEQ ID NO: 11 - DNA, 24 bp, Artificial Sequence
  Description of Artificial Sequencereverse primer for simple sequence length polymorphism (SSLP) marker MUA22
gtcctcatcaaggggctgcagagg

SEQ ID NO: 12 - DNA, 22 bp, Artificial Sequence
  Description of Artificial Sequenceforward primer for simple sequence length polymorphism (SSLP) marker T24H18
cagtgcatggtttgcatgggaa

SEQ ID NO: 13 - DNA, 24 bp, Artificial Sequence
  Description of Artificial Sequencereverse primer for simple sequence length polymorphism (SSLP) marker T24H18
tttacgcaggacatgtttcctctc

SEQ ID NO: 14 - DNA, 20 bp, Artificial Sequence
  Description of Artificial Sequenceforward primer for simple sequence length polymorphism (SSLP) marker T6I14
aaggaccagcgtggctcaag

SEQ ID NO: 15 - DNA, 24 bp, Artificial Sequence
  Description of Artificial Sequencereverse primer for simple sequence length polymorphism (SSLP) marker T6I14
aatcactcactgcctctttggagg

SEQ ID NO: 16 - DNA, 21 bp, Artificial Sequence
  Description of Artificial Sequenceforward primer for simple sequence length polymorphism (SSLP) marker MXE10
cgtcagggtgctgcttttctc

SEQ ID NO: 17 - DNA, 23 bp, Artificial Sequence
  Description of Artificial Sequencereverse primer for simple sequence length polymorphism (SSLP) marker MXE10
gtgcctgcacattgatcaccatc

SEQ ID NO: 18 - DNA, 26 bp, Artificial Sequence
  Description of Artificial SequencePCR amplification primer for ABO1 2,173 bp promoter fragment pABO1 to add cacc at the 5′ end
caccggaaggaagagagctgaagggc

SEQ ID NO: 19 - DNA, 23 bp, Artificial Sequence
  Description of Artificial SequencePCR amplification primer for ABO1 2,173 bp promoter fragment pABO1
gagggtcagagggattcagaagg

SEQ ID NO: 20 - DNA, 21 bp, Artificial Sequence
  Description of Artificial Sequence967 bp RD29Afragment PCR amplification forward primer
gacgagtcaggagctgagctg

SEQ ID NO: 21 - DNA, 22 bp, Artificial Sequence
  Description of Artificial Sequence967 bp RD29Afragment PCR amplification reverse primer
cgatgctgccttctcggtagag

SEQ ID NO: 22 - DNA, 24 bp, Artificial Sequence
  Description of Artificial Sequence552 bp RD29Bfragment PCR amplification forward primer
ccgacgggaactcatgatcagttc

SEQ ID NO: 23 - DNA, 23 bp, Artificial Sequence
  Description of Artificial Sequence552 bp RD29Bfragment PCR amplification reverse primer
cacttccacctcctttgtagccg

SEQ ID NO: 24 - DNA, 22 bp, Artificial Sequence
  Description of Artificial Sequence413 bp COR47fragment PCR amplification forward primer
gaagctcccaggacaccacgac

SEQ ID NO: 25 - DNA, 21 bp, Artificial Sequence
  Description of Artificial Sequence413 bp COR47 fragment PCR amplification reverse primer
cagcgaatgtcccactcccac

SEQ ID NO: 26 - DNA, 22 bp, Artificial Sequence
  Description of Artificial Sequence517 bp ABI1 fragment PCR amplification forward primer
cgcaggtcctttcaggccattc

SEQ ID NO: 27 - DNA, 20 bp, Artificial Sequence
  Description of Artificial Sequence517 bp ABI1 fragment PCR amplification reverse primer
gccatggccgtcgtaaacac